(19) United States
(12) Patent Application Publication
(10) Pub. No.: US 2023/0240696 A1
(43) Pub. Date: Aug. 3, 2023

(54) CLOT REMOVAL DEVICE AND METHOD OF USING SAME

(71) Applicant: Hesham Morsi, Houston, TX (US)

(72) Inventor: Hesham Morsi, Houston, TX (US)

(21) Appl. No.: 17/591,047

(22) Filed: Feb. 2, 2022

(51) Int. Cl. A61B 17/221

(52) U.S. Cl. CPC A61B 17/221; A61B 2017/00778

(57) ABSTRACT

A minimally invasive endovascular device for treating a blocked or obstructed biological lumen, such as a blood vessel fully or partially obstructed by deposits of biological matters in or non-biological matters. Certain embodiments of the present disclosure comprise two capture members that are configured to be placed on either side of the obstruction and enclose around the obstruction for removal. Embodiments of the present disclosure also provide methods for implementing an endovascular device according to aspects of the present disclosure.

### TECHNICAL FIELD

Embodiments of the present disclosure relate generally to medical devices and methods of using same, and more specifically, to medical devices for treating or removing an obstruction material from an obstructed or occluded biological lumen, such as an embolus or clot in a blood vessel.

### BACKGROUND

This section is intended to introduce various aspects of the art, which may be associated with exemplary embodiments of the present disclosure. This discussion is believed to assist in providing a framework to facilitate a better understanding of particular aspects of the present disclosure. Accordingly, it should be understood that this section should be read in this light, and not necessarily as any admission of prior art.

There are many reasons a blood vessel becomes blocked or obstructed. One common way is from deposition of thrombus or clot inside the lumen of the blood vessels, which can restrict the antegrade blood flow through the lumens of these blood vessels to or out of one or more body tissues. Because arterial blockages reduce blood flow through the affected vessel, any blockage or obstruction can lead to many serious medical complications as tissue relying on the blood's supply of oxygen may become damaged due to the decrease in the oxygen amount. For instance in the brain circulation this can lead to stroke with loss of vital brain functions and/or death, while in the heart it can lead heart attack due to damage of the heart muscle with significant impairment of its ability to pump blood to the body organ, which can eventually lead to death. Venous clots and blockage on the other hand can lead to deleterious effects such as interference with venous blood flow out of the body tissues leading to venous congestion and tissue edema which can compress vital structure and interfere with its function, progressing to venous hypertension with eventual rupture of the thin walled weaker veins and tissue hemorrhage or interference with antegrade arterial flow which can lead to tissue ischemia and death. Lastly a venous clot in the pulmonary circulation, such as a Pulmonary Embolism (PE), can interfere with circulation of blood from the heart to the lung for oxygenation which can results in severe chest pain and sudden death.

While various methods and devices are available to treat a blockage or obstruction through removal of the obstructing clot, these devices can be traumatic to the blood vessel due to stiffness and pressure exerted on the lining of the vessel during clot engagement and removal. In addition, they usually do not sufficiently capture and retain the particulate matters from the obstruction. In particular, these methods usually cause fragmentation of the clot either during device engagement of the clot or due to the friction between the not fully encapsulated clot with the wall of the vessels or the flowing blood during the removal from the body. This fragmentation of the clot can lead to migrating of clot fragments with the blood flow either to the same treated blood vessels and its branches distal to the original obstruction site or to another unaffected area of the vascular system in the branching circulation at or proximal to the site of the obstruction where they can get lodged again and causes further obstruction. There remains a need for new devices to safely remove the obstructing clot fully encapsulated and shielded from the friction force with the blood vessel wall and the flowing blood to prevent excessive fragmentation and distal migration. Additionally, while current technologies represent significant improvement in removing clots from circulatory system and restoration of blood flow, significant limitations exists including the limitation of reliance on clot consistency and the need for several passes and attempts to remove the obstruction, which can increase the time for effective revascularization. Additionally, current technologies may require the concurrent use of other techniques and devices to improve the rate of effective revascularization, such as suction and flow arrest using large bore catheter and balloon guide catheters, which also can also add to the complexity of the procedures and potential risks. Thus, a critical need remains for new devices for effectively removing the clot mostly or entirely in a single pass regardless of clot consistency. Devices with reduced risk of fragmentation and distal migration, and reduced trauma to the blood vessel walls and without the need for adjunct devices and techniques such as flow arrest, are also needed.

### BRIEF SUMMARY

Embodiments of the present disclosure provide a minimally invasive endovascular device for treating a blocked or obstructed biological lumen, such as a blood vessel fully or partially obstructed by deposits of biological matters or non-biological matters. Certain embodiments of the present disclosure comprise two capture members that are configured to be placed on either side of the obstruction and enclose around the obstruction for removal. Embodiments of the present disclosure also provide methods for implementing an endovascular device according to aspects of the present disclosure.

According to aspects of the present disclosure, a device to remove an obstruction in a lumen is described. In some embodiments, the device comprises a first capture member and a second capture member, where each capture member comprises an open end and a tapered end. In some embodiments, the open end is defined by a frame component coupled to a body component, the body component extending between the open end and the tapered end. The frame component may be a ring frame component, which may self-expand. The frame component may have a leading notch, which may be a protrusion. The leading notch may be designed to act as a leading lip or spine to first engage and dislodge an obstruction. In some embodiments, the leading notch of the first capture member is configured to interlock with the leading notch of the second capture member. The device may further comprise a first guide member and a second guide member. The first guide member may be directly coupled to the first capture member. The first guide member may be coupled, such as at the open end or the tapered end. In some embodiments, at least a portion of the body component is attached to the first guide member. The second guide member coupled to the second capture member. The second guide member may be coupled, such as at the open end or tapered end. In some embodiments, at least a portion of the body component is attached to the second guide member. In some embodiments, the first guide member and the second guide member are slidably coupled to each other. In some embodiments, the first guide member is disposed in the second guide member or vice versa.

In some embodiments, the frame component of the first capture member and/or the second capture member is at a non-perpendicular angle to the first guide member and/or the second guide member, respectively. The angle may be fixed. The angle may be greater than 90 degrees, such as between 90 degrees and 120 degrees. In some embodiments, the angle is 91 degrees, 92 degrees, 93 degrees, 94 degrees, 95 degrees, 96 degrees, 97 degrees, 98 degrees, 99 degrees, 100 degrees, 101 degrees, 102 degrees, 103 degrees, 104 degrees, 105 degrees, 106 degrees, 107 degrees, 108 degrees, 109 degrees, 110 degrees, 111 degrees, 112 degrees, 113 degrees, 114 degrees, 115 degrees, 116 degrees, 117 degrees, 118 degrees, 119 degrees, or 120 degrees.

In some embodiments, the frame component comprises a self-expanding ring. The self-expanding ring may expand the open end of the frame component. The frame component, including the self-expanding ring, may be any shape capable of performing the designed purpose of removing an obstruction in a lumen. The frame component may be shaped to fit the dimensions of a lumen, including an obstructed lumen. In some embodiments, the frame component is substantially circular or elliptical. In some embodiments, the frame component is rectangular shaped.

In some embodiments, a measurement, such as the frame component's diameter and/or height, of the first capture member and/or the second capture member is approximately 0.1 mm, 0.2 mm, 0.3 mm, 0.4 mm, 0.5 mm, 0.6 mm, 0.7 mm, 0.8 mm, 0.9 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, or larger. In some embodiments, the measurement corresponds to a particular lumen structure, such as one or more venous or arterial structures. The diameter may be any measurement going through the center of the frame component. The height may be measured perpendicularly from where the capture member is connected to the guide member to a point parallel with the leading notch. In some embodiments, the measurements of the frame component(s) are fixed and preselected to match the circumference of a lumen, such as an obstructed lumen. In some embodiments, measurements of the frame component(s) are slightly larger than the circumference of an obstructed lumen.

In some embodiments, the leading notch of the first capture member and/or the second capture member is approximately 1×1 mm, 1.1×1 mm, 1.2×1 mm, 1.3×1 mm, 1.4×1 mm, or 1.5×1 mm. In certain embodiments, the measurements of the leading notch are selected depending on the size of the corresponding frame component. In some embodiments, the leading notch is approximately 10-30% of the circumference of the frame. In some embodiments the depth of the leading notch is approximately 50-150% of the width of the leading notch.

Certain embodiments concern a device comprising a first capture member, a second capture member, and a guide member. The first capture member and the second capture member may comprise a body component, including an open end comprising a frame component, the frame component may be configured to be angled toward an obstruction, and a leading notch extending from the frame component toward an obstruction. The leading notch may be parallel to the guide member. In some embodiments, the first capture member and the second capture member are connected to its respective guide member and configured to mechanically separate an obstruction in a lumen. In some embodiments, the device is configured to mechanically separate an impacted or adherent clot from the walls of a blood vessel. The first capture member and the second capture may be capable of being manipulated with the guide member to contact the obstruction. The contact may be via the frame components of both capture members. In some embodiments, the obstruction is first contacted with the leading notch.

In certain embodiments, the device is configured to encapsulate an obstruction with in at least one body component of the device.

In another embodiment, the frame component is configured to fit within a catheter for delivery to the obstruction and to expand to define the open end when released from the catheter, the catheter having a radius smaller than a radius of the open end. In one embodiment, the frame component comprises a self-expanding material, such as nitinol, configured to have an original configuration and a deformed configuration; where the self-expanding material is configured to change from the deformed configuration to the original configuration at least by exposure to an activating condition. The activating condition may occur when the frame component is released from a constraint, such as release from the constraint of a catheter. The activating condition may be achieved through mechanical means. For example, the activation may be done using a dial mechanism, which may be manually or electronically controlled. In one embodiment, the diameter of the frame at open end can be adjusted up or down through a dial mechanism that can be manually or electronically controlled.

The frame component may comprise a wire having a thickness between 0.003 inches to 0.010 inches. In some embodiments, the frame component comprises a hollow wire, such as a hollow nitinol wire, with an inner core diameter of 0.001-0.004 inches. In another embodiment, the deformed configuration allows the device to fit within the catheter.

In one embodiment, the device further comprises a radiopaque material. In one embodiment, visualization of the frame component under x-ray is aided by at least one radio-opaque marker attached to the frame component, the leading notch, or the guide member at or close to the attachment with the frame component. In another embodiment, visualization of the frame component under x-ray is enhanced by a wire capable of being detected by an external detection system. The wire may be a radiopaque microwire of radiopaque material, such as platinum, disposed inside the core of the frame component and/or the leading notch. In some embodiments, the frame component and/or the leading notch are comprised of a radiopaque material. In another embodiment, visualization of the frame under x-ray is enhanced by a radiopaque wire wrapped around the outer surface of the frame component and/or the leading notch.

In one embodiment, the body component is attached to the respective guide member substantially along the length of the body component. In another embodiment, at least one capture member further comprises a support arm coupled to the respective guide member and the respective frame component. In one embodiment, the support arm comprises a first end and a second end, where the first end is coupled to the respective frame component and the second end is coupled to the respective guide member. In another embodiment, the second end is attached to the respective guide member. In another embodiment, the respective guide member further comprises a fastening member slidably coupled to the respective guide member and where the second end is attached to the fastening member.

In one embodiment, the body component is fluid impermeable. In another embodiment, the body component comprises a woven material of at least one of the following: a polymer, a metal, and any combination thereof. In another embodiment, the body component covers at least a portion of a surface of the frame component to which the body component is coupled. In another embodiment, at least one guide member is adapted to provide a suctioning force. In one embodiment, the body component comprises a non-fenestrated, fluid impermeable sleeve extending between the taper end attachment to the guide member and the open end attachment to the frame component. In some embodiments, the sleeve is made from flexible polymer material such as polyethylene terephthalate (PET) and/or nylon. In another embodiment, the body component is fluid permeable through micro-fenestrations along at least part of the sleeve. This may allow blood to flow through the body component but retain clot fragments inside the body of capture member.

In one embodiment, the total length of each capture member, including when deployed, is between about 5 mm and about 50 mm. In certain embodiments, such as for arterial obstructions, the total length of both capture members, when interlocked, is between about 10 mm and about 30 mm. In another embodiment, the total length of both capture members, when interlocked, is between about 10 mm and 60 mm. In another embodiment, such as for venous obstructions, the total length of both capture members, when interlocked, is about 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm.

In one embodiment, the body component attaches to at least a portion of a surface of the frame component to which the body component is coupled. In another embodiment, the body component attached to and fully cover the surface of the frame. In another embodiment, the body component attaches to at least a portion of a surface of the leading notch. In another embodiment, the body component attaches to and fully covers the surface of the frame component and the leading notch.

In one embodiment, a surface of the body component contains material to enhance the movement of the body surface over the surface of adjacent lumen, including the obstruction. Example material includes a hydrophilic material and/or coating.

In one embodiment, the diameter of one frame component is smaller than the diameter of the other frame component. In certain embodiments, such as for use in a tapering blood vessel, the diameter of the frame component of a first capture member, such as the capture member distal to an obstruction, is smaller than the diameter of the frame component of the second capture member and is configured to be retrieved inside the frame of the second capture member by manipulation of the guide members to form a tight seal enclosure around the obstruction and unite the capture members in interlocking fashion.

In other embodiments, the frame component of one capture member and at least a portion of the capture member body are configured to be retrieved inside the frame component and at least a portion of the body of the other capture member by manipulation of the guide members to form a tight seal enclosure around the obstruction and unite the capture members in interlocking fashion. Interlocking of the capture members may involve the withdrawal of one leading notch partially or fully underneath the other leading notch and/or the pushing of one leading notch partially or fully over the other leading notch. Interlocking of the capture members may additionally involve the withdrawal of one frame component partially or fully underneath the other frame component and/or the pushing of one frame component partially or fully over the other frame component. Additionally, interlocking of the capture members may involve the withdrawal of one leading notch and its corresponding frame component underneath and proximal to the other leading notch and its corresponding frame component or the pushing of one leading notch and its corresponding frame component over and distal to the other leading notch and its corresponding frame component to form a tight seal around the obstruction.

In certain embodiments, once the capture members are interlocked and united in a tight seal around an obstruction, the capture members are locked together with the obstruction trapped inside in this configuration during the removal process by pinning the two guide members firmly together. The guide members may be pinned in place using a hand-held docking station attached to both guide members. The docking station may or may not have a locking mechanism.

In another embodiment, at least one frame component comprises an inflatable member. In another embodiment, the device further comprises an inflatable member disposed near at least one frame component.

In one embodiment, at least one guide member comprises a body with a channel disposed therein, the body having at least one aperture positioned near an end of the respective capture member at or close to the obstruction. In one embodiment, the guide member is used to provide a suctioning force. In one embodiment, the guide member is used to deliver a therapeutic substance to the obstruction. The guide member comprises at least one channel and/or aperture for delivering the therapeutic substance. In some embodiments the channel(s) and/or apertures are located on or near either body components. In some embodiments, the therapeutic substance, when delivered, remains contained within one or both of the body components. In another embodiment, the coupling between at least one guide member and the respective frame member comprises the at least one guide member attached to an outer surface of the respective frame member.

In another embodiment, the angle of attachment of each frame component to its corresponding guide member and/or the angle of attachment of the leading notch to its corresponding frame component can be manually adjusted and varied together or separately through mechanical manipulation to allow for optimal angles of engagement with an obstruction, including in tortuous non-linear anatomy of the vessel walls. Mechanical manipulation can be achieved, for example, through one or more micro-cables running through or alongside the corresponding guide member and attached to the corresponding frame component and/or leading notch, including at one or more hinge joints. In other embodiments, mechanical manipulation of the frame component and/or the leading notch in repeating alternating directions allow for juggling movement on the adjacent obstruction surface and may be used to help dislodge and separate the obstruction from the lumen wall.

In other embodiments, the size and/or shape of the frame component and/or the leading notch is manually adjusted and/or varied to fit variable anatomies and obstructions. Mechanical manipulation can be achieved, for example, through one or more micro-cables running through or alongside the corresponding guide member and attached to the corresponding frame component and/or leading notch at one or more hinge joints. Mechanical manipulation can also include a dial mechanism.

In one embodiment, visualization of the obstructed lumen, vessel wall, and/or obstruction is enhanced in situ through the incorporation of one or more micro-transducers. The micro-transducer may be coupled to the leading notch and/or the frame component to produce ultrasound or optical tomography imaging. In other embodiments, energy waves can be produced from the micro-transducers at the frame component and/or leading notch. In some embodiments, the energy waves are precisely direct toward an obstruction surface, which may help in optimal encapsulation and removal of the obstruction. Targeted energy waves can for example be used to alter an obstruction's organization and consistency leading to, for example, clot shrinkage and separation from vessel wall.

In another embodiment, the frame component has two or more leading notches disposed around the circumference of the frame component. In another embodiment, the frame component comprises two or more rings of self expanded wires attached to the body component and connected to the guide member at similar or different angles, forming a collar to expand the open end. In some embodiments, the ring closer to the obstruction has one or more leading notches.

According to another aspect of the present disclosure, there is provided a method for removing an obstruction in a lumen. The method comprises the steps of delivering an endovascular device to the location of the obstruction in a patient using a catheter, where the device comprises a first capture member and a second capture member, where each capture member comprises an open end and a tapered end, where the open end comprises a frame component coupled to a body component, the body component extending between the open end and the tapered end. In some embodiments, the device further comprises a first guide member coupled to the first capture member at the open end and along at least a portion of the body component, and a second guide member coupled to the second capture member at the open end and along at least a portion of the body component, where the first capture member and the second capture member are slidably coupled to each other, the slidable coupling comprises the first guide member disposed in the second guide member. In some embodiments, the frame component is attached to corresponding guide member at fixed angle giving the open end of each capture member a slanted appearance toward the obstruction. In some embodiments, each frame component has at least one leading notch facing toward the obstruction and is attached to the frame component at a fixed angle. The leading notch may be parallel, or approximately parallel to the guide member.

In some embodiments, the method further comprises the steps of positioning a distal end of the catheter distal to the obstruction, withdrawing the catheter to release the first capture member distal to the obstruction; positioning the distal end of the catheter proximal to the obstruction; withdrawing the catheter to release the second capture member proximal to the obstruction; enclosing the obstruction with the capture members by manipulating at least one of the first guide member and/or the second guide member to retrieve the first capture member frame component and leading notch under and inside or proximal to the second capture member frame component and leading notch to interlock the first capture member partially or fully inside the second capture member, effectively trapping the obstruction inside the enclosed space of the united capture members; and removing the captured obstruction by removing the interlocked capture members from the lumen and into a proximal guiding catheter.

In one embodiment, the method further comprises the step of providing a suctioning force through at least one guide member. In another embodiment, the method further comprises the step of repeatedly moving at least one leading notch and/or frame component against the obstruction. In another embodiment, the method further comprises the step of delivering a therapeutic substance to the obstruction. In one embodiment, the delivering is achieved at least through one of the guide members, the guide member comprising a body with a channel or vessel disposed therein, the body having at least one aperture positioned near an end near the respective capture member.

In one embodiment, the enclosing step comprises manipulating the first guide member to engage a surface of a leading notch and/or frame component of the first capture member with a surface of the obstruction while the second guide member is maintained stationary. In another embodiment, the enclosing step comprises manipulating the second guide member to engage a surface of a leading notch and/or frame component of the second capture member with a surface of the obstruction while the first guide member is maintained stationary. In another embodiment, the enclosing step comprises manipulating both the first and second guide members to engage a surface of a leading notch and/or frame component of both capture members with a surface of the obstruction.

The foregoing has outlined rather broadly the features and technical advantages of the embodiments of the present disclosure in order that the detailed description of these embodiments that follows may be better understood. Additional features and advantages of the embodiments of the present disclosure will be described hereinafter which form the subject of the claims of the present disclosure. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the spirit and scope of the present disclosure as set forth in the appended claims. The novel features which are believed to be characteristic of the present disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the embodiments of the present disclosure, reference is now made to the following descriptions taken in conjunction with the accompanying drawing, in which:

FIGS. 1A-1C illustrate side perspective views of certain exemplary stages in an exemplary embodiment of a procedure for using a first embodiment of the endovascular device according to certain aspects of the present disclosure.

FIG. 2 illustrates a perspective view of the first embodiment of the endovascular device according to certain aspects of the present disclosure in a closed configuration traveling through a blood vessel; and

FIG. 3 illustrates a perspective side view of the first embodiment of the endovascular device according to certain aspects of the present disclosure in a closed configuration entering a catheter;

FIGS. 4A-4D illustrate side perspective views of certain exemplary stages in an exemplary embodiment of a procedure for using a second embodiment of the endovascular device according to certain aspects of the present disclosure;

FIG. 5 illustrates a perspective side view of the second embodiment of the endovascular device according to certain aspects of the present disclosure in a closed configuration entering a catheter;

FIGS. 6A and 6B illustrate side perspective views of a third embodiment of capture members of the endovascular device according to certain aspects of the present disclosure;

FIG. 7 illustrates a side perspective view of the capture members of FIG. 6A coupled to one another; and

FIGS. 8A-8F are side perspective views of certain exemplary stages in an exemplary embodiment of a procedure for using the endovascular device shown in FIG. 7 according to certain aspects of the present disclosure.

FIG. 9 shows an embodiment comprising two capture members that are configured to be placed on either side of the obstruction and enclose around the obstruction for removal.

FIG. 10 shows an embodiment comprising a first capture member with a frame component and a second capture member with a frame component angled in opposing directions.

FIG. 11 shows an example of the frame component, including ways to measure the frame component.

FIG. 12 shows the interaction of the frame components and leading notches between the first capture member and the second capture member.

FIG. 13 shows the interaction of the leading notches and the slanted angled frame components of each capture member.

FIG. 14 shows the smooth retrieval of the capture members and interlocked leading notches.

FIG. 15 shows an embodiment where the frame components are angled in the same direction resulting in both frame components being parallel to each other.

FIG. 16 shows embodiments where the leading notch and frame components are in different configuration and/or manually manipulated.

FIG. 17 shows an example of mechanism to manually manipulate and adjust the angels of the notch and/or the frame components through one or more microcables.

FIG. 18 shows an embodiment comprising the incorporation of at least one micro-transducer.

FIG. 19 shows an embodiment where the body component covers the leading notch on both capture members.

FIG. 20 shows a self-expanding skeleton for a body component.

It should be understood that the drawings are not necessarily to scale and that the disclosed embodiments are sometimes illustrated diagrammatically and in partial views. In certain instances, details which are not necessary for an understanding of the disclosed methods and apparatuses or which render other details difficult to perceive may have been omitted. Also, for simplification purposes, there may be only one exemplary instance, rather than all, is labeled. It should be understood, of course, that this disclosure is not limited to the particular embodiments illustrated herein.

### DETAILED DESCRIPTION

Embodiments of the present disclosure provide for minimally invasive, effective, and safe removal of an obstruction, which may be an obstructing material, such as a clot or embolism, disposed in a patient's (human or otherwise) vascular system. The obstruction may be in any tubular structure with a lumen, such as a blood vessel. The blood vessel may be an artery transporting blood to human tissue or a vein transporting blood from human tissue. The obstruction may be impacted in the lumen and/or adherent to a wall of the lumen, which may result in complete or partial obstruction of flow through the lumen. Certain embodiments of the present disclosure are applicable for extraction of material in small, tortuous and highly branching segments of the neurovascular system. Certain embodiments are applicable for extraction of material in the arterial system of the brain, including for effective treatment of ischemic stroke and timely restoration of antegrade blood flow to brain tissue. Other embodiments of the present disclosure are applicable for extraction of clot and embolus from other body arterial systems, including the heart circulation, for effective timely restoration of antegrade blood flow to heart muscle and nerves. Other embodiments of the present disclosure are applicable for extraction of clots from body venous systems, including, brain venous sinuses, pulmonary circulation, and peripheral veins.

In a general embodiment, the endovascular device of the present disclosure includes two opposing capture members that are slidably coupled to each other. Each capture member preferably comprises an open end and a tapered end, where the open end of each capture member faces one another. The open ends may be angled towards or away from each other.

In some embodiments, the leading notch of a capture member has a fixed parallel, or fixed approximately parallel, angle of attachment to the frame component relative to the guide member to which the frame component is attached. The leading notch may be a 2D shape including but not limited to semicircular, a semi-oval, horse shoe, rectangular, or any other suitable atraumatic shape. The leading notch may have a 3D shape with convexity that follows the contour of the vessel or the frame component from which it arises.

In some embodiments, the angle of attachment of the first frame component to the first guide member is similar to angle of attachment of the second frame component to the second guide member both. In some embodiments, the first frame component is parallel, or approximately parallel, to the second frame component. In some embodiments, the first frame component is perpendicular, or approximately perpendicular, to the second frame component. In certain embodiments, the angle of attachment of first frame to first guide member is different from angle of attachment of second frame to second guide member. In an embodiment, the frame components are angled toward the obstruction giving the open ends at the first and second capture members a slanted appearance toward the obstruction. In other embodiments, the open end of the first capture member is slanted toward the obstruction while the open end of the second capture member is slanted away from the obstruction.

In one embodiment, the endovascular device can be delivered to the site of the material deposit using a catheter. The capture members can be placed on each side of the material deposit with the open ends facing the material deposit. In one embodiment, the open end of each capture member is supported by a frame component. In some embodiments, the frame component comprises a leading edge facing the obstruction and is configured to first approach an obstruction surface and seek a line of separation between the obstruction outer surface and the lining of a wall of the lumen to which the obstruction is attached. In another embodiment, the capture members are slidably coupled to one another to allow the capture members to move in the distal and proximal directions to facilitate in dislodging the clot from the arterial wall. In a preferred embodiment, the majority of the material deposit is moved into the capture members at the site of lodging when the capture members encloses the clot as they progress toward one another. In one closed configuration, the open end of the capture members meet one another to form an enclosure to capture and retain the material deposit contained therein. The capture members can be withdrawn in this closed configuration and/or be pulled into a catheter, thereby removing the material deposit.

Embodiments of the present disclosure provide for obstruction removal without excessive force, pressure, and/or compression of the obstruction, including by enclosing around the obstruction at the site of obstruction without excessive manipulation of the obstruction, thereby minimizing fragmentation of the obstruction or squeezing of the obstruction into side branches that may exist at the site of obstruction, which can lead to further damage. Minimizing excessive force, pressure, and/or compression may also minimize movement and fragmentation of any fragments not contained in the device from migrating away from the original site, such as into smaller branches, which can lead to further damage. Embodiments of the present disclosure also provide for obstruction removal without excessive pressure on the lumen wall by reducing lumen wall contact through elements of the design including, the tapering design of the capture members with maximum size at the open end, the accurate sizing of the open ends of capture members in relations to the vessel walls, and the use of mostly flexible materials, thereby minimizing wall contact and trauma. In some embodiments, trauma is minimized to the endothelium and other tissue in of the blood vessels, which prevents further damage or vessel rupture especially in thin walled veins. Embodiments of the present disclosure also provide for a device for effectively removing the obstruction mostly or entirely in single pass regardless of obstruction consistency.

Embodiments concern a minimally invasive endovascular device for treating a blocked or obstructed lumen, such as a blood vessel with a clot. Embodiments of the present disclosure comprise at least two capture members that are configured to be placed on either side of the obstruction and enclose around the obstruction for removal. Capture members may have opposing open ends slanted toward the obstruction in fixed predetermined angles. They may be expanded by at least one self-expanding frame component. The frame component may have a leading notch in fixed angle and parallel to the vessel wall designed to act as leading edge to first engage and dislodge the obstruction for optimal encapsulation. In certain embodiments, the leading notch and frame components can be manually manipulated to adjust the angles of engagement with the obstruction and/or to mechanically separate adherent obstruction from the vessel wall,

Embodiments of the present disclosure also provide methods for implementing an endovascular device according to aspects of the present disclosure.

In one embodiment, retrieving the united, interlocked capture members and trapped obstruction involve first retrieving the tapered end and body of a first capture member inside the lumen of the guide catheter with the mouth of the guide catheter pushing the distally facing first frame component and leading notch down toward the guide member which in turn push the underneath interlocked proximally facing second frame component and leading notch toward the guide member thus allowing the mouth of the guide catheter to smoothly glide over the overlapping frame components and leading notches without getting caught against the mouth of the guide catheters or interfere with the smooth full retrieval of the united, interlocked capture members and trapped obstruction inside the lumen of the guide catheter and eventually out of the body.

According to another aspect of the present disclosure, there is provided a method for removing an obstruction in a lumen. The method comprises the steps of delivering an endovascular device to the location of the obstruction in a patient using a catheter, where the device, in some embodiments, comprises a first capture member and a second capture member, where each capture member comprises an open end and a tapered end, where the open end comprises a frame component, comprising a leading notch, coupled to a body component, the body component extending between the open end and the tapered end. In some embodiments, the device further comprises a first guide member coupled to the first capture member at the open end and tapered end, and a second guide member coupled to the second capture member at the open end and tapered end, where the first capture member and the second capture member are slidably coupled to each other, the slidable coupling comprises the first guide member disposed in the second guide member. In some embodiments, the frame component is attached to corresponding guide member at manually adjustable angle. In some embodiments, the frame component has a leading notch facing toward the obstruction and is attached to the frame component also at manually adjustable angle, in some embodiments. Manually adjusting the fame component and/or leading notch angles may allow for better engagement of the obstruction surface and may even allow for the dislodgment of impact adherent obstruction by repeated tapping motion. In some embodiments, the method further comprises one or more of the steps of positioning a distal end of the catheter distal to the obstruction, withdrawing the catheter to release the first capture member distal to the obstruction; positioning the distal end of the catheter proximal to the obstruction; withdrawing the catheter to release the second capture member proximal to the obstruction; enclosing around the obstruction with the capture members by manipulating at least one of the first guide member and/or the second guide member to retrieve the distal frame component and leading notch under and inside or proximal to the proximal frame component and leading notch to unite and lock the distal capture member partially or fully inside the proximal capture member, effectively trapping the obstruction inside the enclosed space of the united, interlocked capture members; and removing the captured obstruction by removing the united, interlocked capture members from the lumen.

In another embodiment, the body components, frame components and/or leading notches comprise a self-expanding material, including any such metal, such as nitinol, or any such polymer, and any combination thereof. In some embodiments, the material is braided. In another embodiment, the body component, frame component, and/or leading notches of the first capture member is made from material different from the body component, frame component, and/or leading notches of second capture member. For example, the body component of the first capture member may comprise a non-fenestrated, fluid-impermeable sleeve made from flexible polymer material such as PET, while the body component of the second capture member comprises a braided, self-expanding material of a metal such as nitinol. In certain embodiments with one or more body components made of braided self expanded nitinol material, the frame component may not be needed as the self-expanded end of the braid attached to the open end can support and expand the open end of the capture member to its full size.

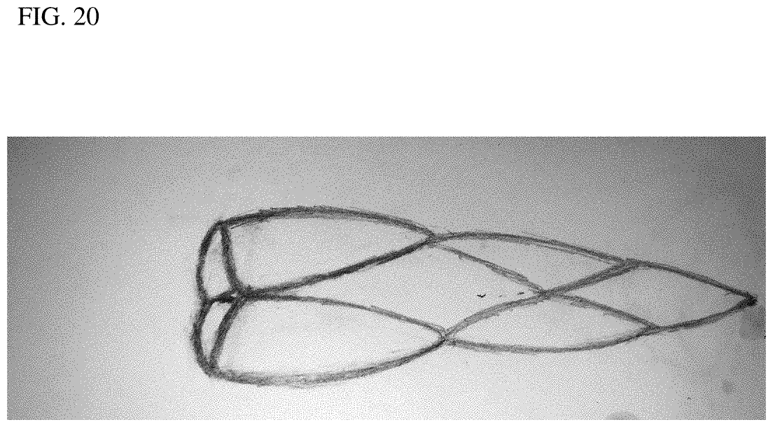

In embodiments with one or more body components made of flexible polymer such as PET or nylon, full expansion of the body component can be aided by one or more wires of self-expanding nitinol material coupled to the inner surface of the body in longitudinal, circular, helical, or loosely braided configuration and any combination thereof. In embodiments with one or more body components made of flexible polymer such as PET or nylon, full expansion of the body component can be aided by a skeleton of self expanded braided nitinol wires that are disposed within the body and generally has the same shape and size of the body of the capture member, such as shown in FIG. 20. The skeleton is attached to the body and guide member at the open end and or tapered end.

Throughout the specification, there are references a “first” and a “second” aspect of the device, such as a “first body member” or a “second frame component”. However, a reference to a “first” aspect in one description of an embodiment does not necessarily mean that aspect is also a “first” aspect in another description. For example, reference to “a first frame component” may describe a frame component that is distal to an obstruction in one embodiment while also describing a frame component that is proximal to an obstruction in another embodiment.

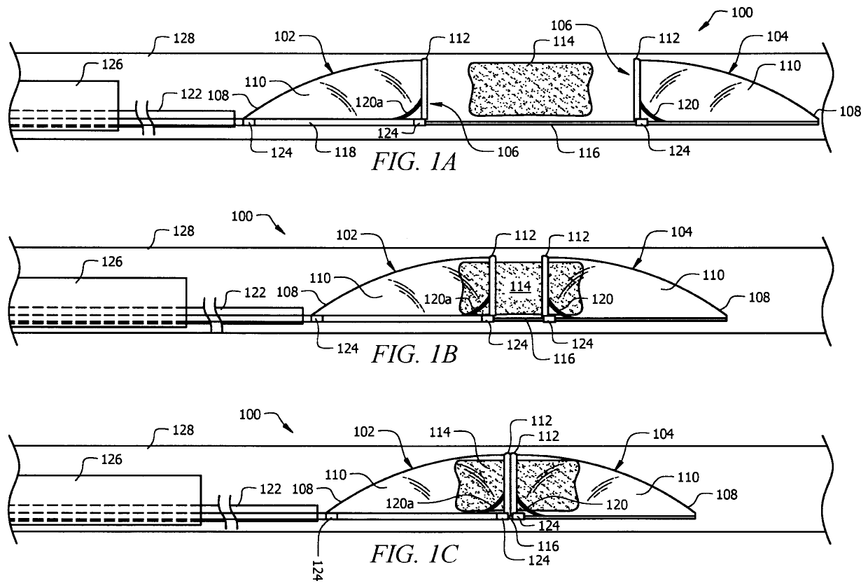

FIGS. 1-3 show certain specific embodiments according to the aspects of the present disclosure. FIGS. 1A-1C show capture members 102 and 104 of endovascular device 100 in a fully expanded configuration. Capture member 102 is the proximal capture member while capture member 104 is the distal member with respect to obstruction 114. In one embodiment, capture members 102 and 104 substantially resemble each other, each comprising open end 106, tapered end 108, and body component 110 extending between open end 106 and tapered end 108. Open end 106 preferably comprises open end support member or frame component 112 defining the shape and size of open end 106 when capture members 102 and 104 are expanded. Frame component 112 preferably has a circular shape that matches the shape of the target blood vessel. In other embodiments, however, frame component 112 can have any shape in the expanded configuration desired, such as circular, oval, rectangular or any other regular or irregular shapes that may be suitable to the particular application.

In one embodiment, frame component 112 comprises a self-expanding material including, but not limited to, a metal, an alloy, a composite, a polymer, and the like. In one non-limiting example, frame component 112 comprises nitinol, stainless steel, cobalt chromium, platinum, titanium, plastic, or any combination thereof. In another embodiment, frame component 112 comprises a superelastic and/or self-expanding material with properties that allow it to have a deformed shape under one condition and to recover its original shape prior to deformation, which can also be referred to as an expanded configuration. A non-limiting example is a memory-shaped heated alloy such as nitinol, or nickel titanium, which is a metal alloy of nickel and titanium. Nitinol alloys exhibit two closely related and unique properties: shape memory and superelasticity. Shape memory refers to the ability of nitinol to undergo deformation at one temperature, then recover its original, un-deformed shape upon heating above its “transformation temperature.” That is, nitinol alloy has a biased expanded condition and may be compressed into a collapsed or deformed condition before use. During use, it may be exposed to temperature above the transformation threshold, thereby causing it to revert back to its un-deformed and/or original shape. Frame component 112 can also comprise any flexible and/or elastic material that allows frame component 112 to be compressed, or deformed by a radial force, to fit into a catheter, such as catheter 122, without sustaining any damage and revert back to its original shape once released from the catheter.

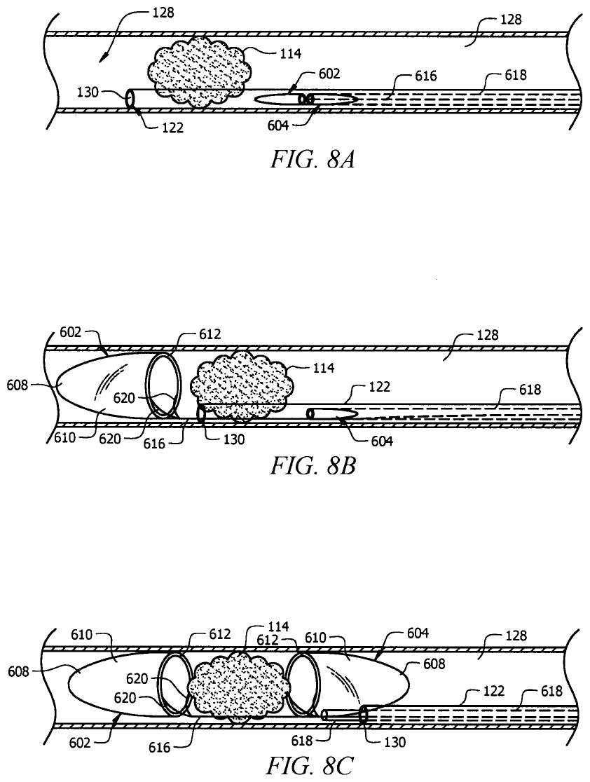

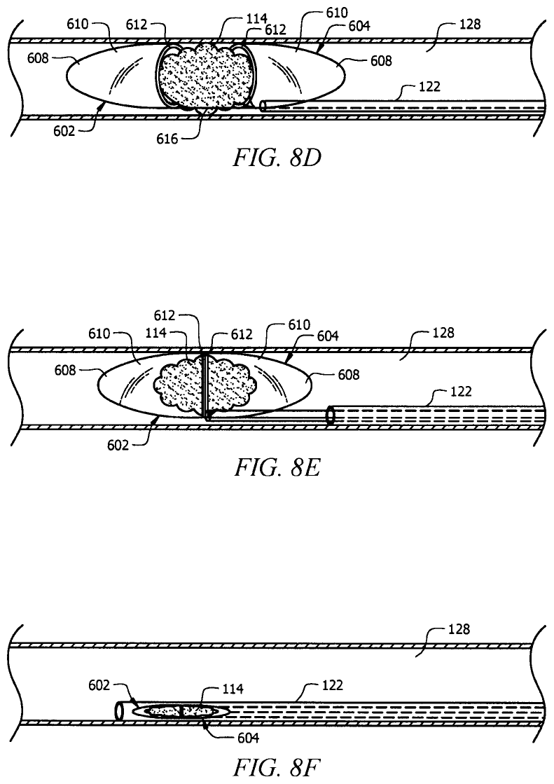

In one embodiment, such as that shown in FIGS. 8A-8B, frame component 112 has a deformed or compressed shape with a smaller diameter than the un-deformed, expanded shape shown in FIGS. 1A-1C. In one embodiment, the thickness of frame component 112 is in a range between about 10 microns to 500 microns. In a preferred embodiment, the thickness of frame component 112 is in a range between about 80 microns to about 120 microns. In another preferred embodiment, the thickness of frame component 112 is in a range between about 95 microns to about 105 microns.

Referring to FIGS. 1A-1C, in a preferred embodiment, the diameter across frame component 112 in an expanded configuration, and thus open end 106, is configured to substantially match the diameter of the particular lumen or blood vessel of interest in which the obstruction, e.g., clot 114, is disposed. In such an expanded configuration, frame component 112 preferably contacts the inner wall of the target blood vessel gently, e.g., without exerting significant force that can damage the blood vessel. This allows at least one capture member 102 or 104 to extend across the interior, or lumen, of the blood vessel where effectively most or all obstructing materials are directed through the respective extended capture member 102 or 104. In one embodiment, such as for arterial applications, the diameter of open end 106 is in a range of about 1.5 mm to about 6 mm, and preferably in a range of about 2 mm to about 4.5 mm. In another embodiment, the diameter of open end 106 is between about 2.5 mm and about 3 mm. In another embodiment, such as for venous applications, the diameter of open end 106 is in a range of about 2 mm to about 20 mm, including in a range of about 8 mm to 10 mm. In general, the measurements of the open ends (which may represent the maximum diameter size of the capture members) are preselected to match the circumference of the intended blood vessel lumen. In other embodiments measurements of the frames are slightly larger than the circumference of the intended blood vessel lumen, for example between 0.1-1 mm.

It is understood that other embodiments can include capture members 102 and 104 of different sizes and configurations. For instance, in one embodiment, one capture member has an open end with a smaller diameter than the other capture member so that one can be inserted into the other, providing an overlapping area. In another embodiment, endovascular device 100 is provided in various sizes and configuration depending on the location of the material deposit to be removed.

Referring to FIGS. 1A-1C, in a preferred embodiment, open end 106 further comprises one end of body component 110 coupled to frame component 112. In such an embodiment, when the respective capture member, 102 or 104, is released from catheter 112, it expands into the configuration shown in FIGS. 1A-1C. In one embodiment, this is achieved with the expansion of frame component 112, which opens body component 110 for material to enter. Body component 110 is preferably formed of any material which is flexible and compatible with bodily tissues and fluids such as blood. In a preferred embodiment, body component 110 is devoid of any fenestration, i.e., the material of body component 110 is impermeable to fluid. Non-limiting examples of suitable materials include polymeric film or fabric-like materials, such as, but not limited to, polyurethane, polyolefin, polyester, plastic, silicone polymers, and any combination thereof. In one embodiment, the material of body component 110 has properties, such as being soft and flexible, that are configured to minimize friction and/or pressure placed on the wall upon contact of the capture members with the lining of the vessel during implementation of device 100. In an alternative embodiment, tapered end 108 can further include at least one fenestration of sufficient size to allow fluids to flow through body component 110 while retaining the captured material deposit. In one embodiment, the material of body component 110 can comprise a material with self-expanding properties as described above, providing it a biased shape in the expanded configuration that allows body component 110 to remain open as it extends away from frame component 112.

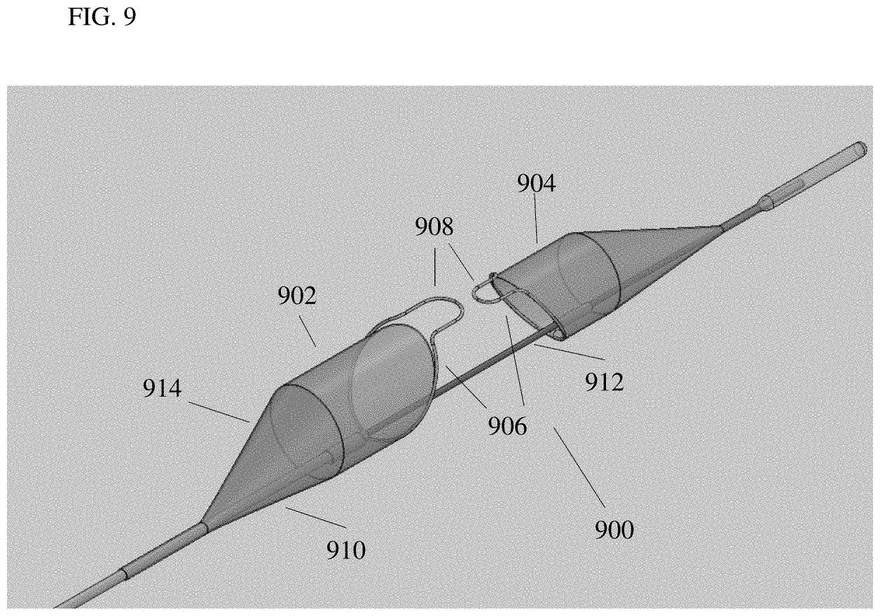

In another embodiment, the body of at least one capture member, such as body component 914 in FIG. 9, comprises a braided, self-expanding material of a metal, such as nitinol, or a polymer, or any combination thereof. In another embodiment, the body component of one capture member is made from material different from the body component of the other capture member. For example, the body component of a first capture member may comprise a non-fenestrated, fluid-impermeable sleeve made from a flexible polymer material, such as PET, while the body component of a second capture member comprises a braided, self-expanding metal, such as nitinol. In embodiments wherein at least one body component is made of a braided, self-expanding material (for example nitinol), a frame component at the open end may not be needed as the self-expanded end of the braid attached to the open end can support and expand the open end of the capture member to its full size.

Body component 110 can be coupled to frame component 112 in any suitable manner. In one embodiment, body component 110 can be attached to frame component at or near the inner diameter or outer diameter of frame component 112. In another embodiment, body component 110 surrounds at least a portion of frame component 112. In such an embodiment, the material of body component 110 contacts the inner wall of the lumen in the expanded configuration instead of frame component 112, which can help protect the inner wall from potential damage or injury resulting from contact with frame component 112 itself. In one embodiment, the body component attaches to at least a portion of a surface of the frame component to which the body component is coupled. Body component 110 can attach to and fully cover the surface of the frame component 112.

Alternatively, expansion of one capture member, 102 or 104, when released from a catheter can be achieved through mechanical means known to those skilled in the art. In one embodiment, frame component 112 comprises an inflatable member comprising an enclosed fillable volume, such as a balloon, that expands when the member is filled with a fluid. In this embodiment, the inflatable member has the shape of frame component 112 as shown, e.g., annular, and body component 110 is coupled to the inflatable frame component. When released from catheter 122, frame component 112 can be expanded by filling the interior of the inflatable member with fluid using methods known to those skilled in the art. The diameter of the inflatable member, and thus, open end 106, can be adjusted based on the amount of fluid provided to the inflatable member. In another embodiment, instead of forming frame component 112 with an inflatable member, frame component 112 is expanded through the expansion of an inflatable member. The inflatable member has a shape that corresponds to the shape of frame component 112 where it can be placed at or near the respective open end 106 so that the radial expansion of the inflatable member pushes against the respective frame component 112 to expand it. Once the respective frame component is expanded, the inflated members can be deflated and removed as appropriate. It is understood that other ways of using an inflatable known to one of ordinary skill in the art can also be used. Other ways can include the addition of self-expanding wire(s) coupled to the inner wall of at least one capture member in circular pattern, longitudinal pattern, helical pattern, or any combination thereof.

Referring to FIG. 1C, when capture members 102, 104 unite to form one capture enclosure, the length of the capture enclosure preferably is longer than the length of the target obstruction, e.g., clot 114. In one embodiment, the total length of both capture members, which may be any capture members described herein, united is between about 5 mm and about 40 mm. In one embodiment, such as for arterial obstructions, the total length of both capture members united is between about 8 mm and about 30 mm, or between about 10 mm and 30 mm. In another preferred embodiment, the total length of both capture members united is between about 10 mm and 12 mm. In yet another embodiment, the total length of both capture members united is about 100 mm.

Referring to FIGS. 1A-1C, capture members 102 and 104 are slidably coupled to one another with the open ends facing each other, allowing them to be moved apart or unite to form one enclosure. In a preferred embodiment, distal capture member 104 is coupled to distal guide member 116, and proximal capture member 102 is coupled to proximal guide member 118. Distal guide member 116 is preferably disposed in proximal guide member 118. In this configuration, the relative position of capture members 102 and 104 can be adjusted in various manners. In one embodiment, a user can hold proximal guide member 118 constant, thereby keeping proximal capture member 102 in one position, while pushing or pulling distal guide member 116 to adjust the position of distal capture member 104. In another embodiment, the user can hold distal guide member 116 constant, thereby keeping distal capture member 104 in one position, while pushing or pulling proximal guide member 118 to adjust the position of proximal capture member 102. In yet another embodiment, both guide members 116 and 118 can be adjusted at the same time to achieve the desired positions of capture members 102 and 104 with respect to each other. Once capture members 102, 104 are in a desired position, that position can be maintained by attaching guide members 116, 118 together, thereby stabilizing endovascular device 100. In one embodiment, distal guide member 116 comprises a solid body, such as a wire; alternative, it can comprise a body with an interior channel, such as a tube. In a preferred embodiment, proximal guide member 118 comprises a tube. In another embodiment, proximal guide member 118 is configured with suction capabilities to assist with bringing clot 114 into capture member 102 and/or 104. In yet another embodiment, both guide members 116, 118 comprise a body with a channel disposed there through having at least one aperture on the body, so guide members 116,118 can be used to provide a suctioning force. Alternatively, or in addition to, the at least one aperture on the body of such guide members in such an embodiment is preferably located near (e.g., at or proximal) clot 114 so that these guide members can also be used to deliver desired substances locally to the site of clot 114. Non-limiting examples of substances that can be delivered include medication configured to facilitate dislodging and removal of clot 114, such as clot dissolving medication that softens and shrinks the clot.

The body of either guide member 116, 118 preferably has a length sufficient to extend through the vascular system of a patient to reach the target accumulation and place endovascular device 100 in the desired deployment location. In one embodiment, either guide member 116, 118 has a length of between about 50 cm and about 250 cm, more preferably a length of about 125 cm and about 175 cm. The diameter of either guide member 116, 118 may be constant or may vary along the length of the respective guide member 116, 118. For example, the diameter of one guide member toward the proximal end away from the user may be between about 0.2 mm and about 1 mm, and preferably about 0.3 mm and about 0.4 mm, while the diameter near the distal end near the clot may be between about 0.05 mm and about 1 mm, and more preferably about 0.1 mm and about 0.2 mm. Accordingly, the diameter of either guide member 116, 118 may taper from the proximal end to the distal end.

Referring to FIGS. 1A-1C, distal capture member 104 is preferably coupled to distal guide member 116 via frame component 112. In a preferred embodiment, frame component 112 is preferably coupled to distal guide member 116 at an angle of about 90 degrees. In one embodiment, the angle between guide member 116 and frame component 112 can be further supported by at least one additional support arm 120, preferably extending between distal guide member 116 and frame component 112. In a preferred embodiment, one end of support arm 120 is coupled to the respective frame component 112 while the other end of support arm 120a is coupled to guide member 116. In another embodiment, one end of support arm 120 is coupled to the respective frame component 112 and the other end is coupled to a fastening component (not shown) slidably coupled to guide member 116, allowing the coupling angle of the respective frame component 112 to be adjusted. In one embodiment, one end of support arm 120 is coupled to distal guide member 116 in a manner that allows it to extend in the proximal direction when capture member 102 is released from catheter 122. In one embodiment, capture member 102 has more than one support arms 120. In another preferred embodiment, body component 110 is attached to distal guide member 116 along at least a portion of the length of body component 110 or only tapered end 108 is coupled to distal guide member 116. In yet another embodiment, body component 110 is coupled to distal guide member 116 from open end 106 to tapered end 108, along the length of body component 110.

In a particular embodiment, each guide member 116, 118 has one attachment site to the outer circumference of its respective frame, thereby leaving substantially all of the respective frame component 112 and open end 106 available for engagement with clot 114. Such a configuration allows for easier transmission of the captured clot inside device 100 through the tortuous paths with minimal interference from guide members 116, 118 or their attachment to frame components 112. Further, this configuration allows the segment of distal guide member 104 to act like a railing upon which clot 114 can move inside capture members 102, 104 when distal capture member 104 is held constant and proximal capture member 102 is pushed.

Proximal capture member 102 is preferably coupled to proximal guide member 118 in a similar manner. In a preferred embodiment, frame component 112 of proximal capture member 102 is preferably coupled to proximal guide member 118 at an angle of about 90 degrees. In one embodiment, the angle between guide member 118 and capture member 104 can be further supported by at least one additional support arm 120a, preferably extending between proximal guide member 118 and frame component 112 of proximal capture member 102. In a preferred embodiment, one end of support arm 120a is coupled to frame component 112 while the other end of support arm 120a is coupled to guide member 118. In another embodiment, one end of support arm 120a is coupled to the respective frame component 112 and the other end is coupled to a fastening component (not shown) slidably coupled to guide member 118, allowing the coupling angle of the respective frame component 112 to be adjusted. In one embodiment, one end of support arm 120a is coupled to proximal guide member 118 in a manner that allows it to extend in the distal direction when proximal capture member 102 is released from catheter 122. In another embodiment, capture member 104 has more than one support arms 120a. In another embodiment, body component 110 is coupled to proximal guide member 118 along at least a portion of the length of body component 110 or only tapered end 108 is coupled to proximal guide member 118. In yet another embodiment, body component 110 is attached to proximal guide member 118 from open end 106 to tapered end 108, along the length of body component 110.

In a particular embodiment, body component 110 of capture members 102 and 104 are configured to fully encapsulate clot 114 and prevent migration of clot 114, thereby reducing the risk of clot 114 from unintentionally ending up at another location in the patient's body. In one embodiment, this is achieved by forming body component 110 of suitable materials do not have any fenestration.

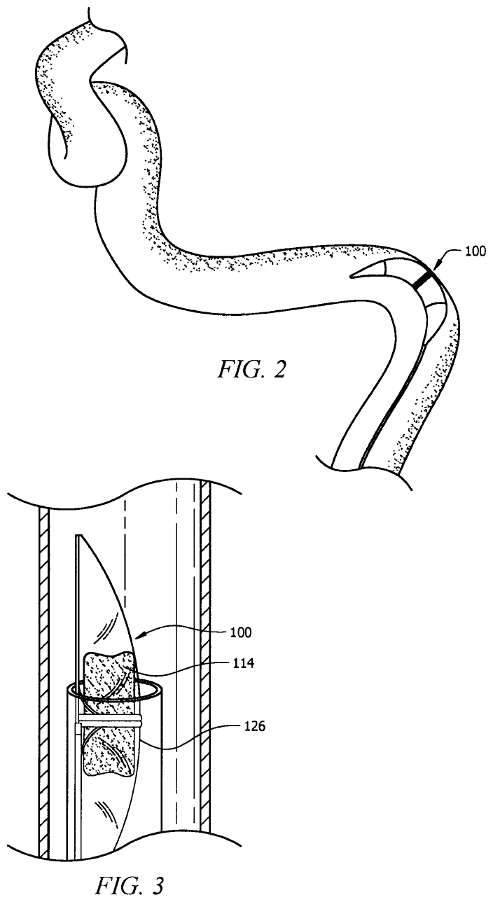

In a particular embodiment, endovascular device 100 includes at least one radiopaque portion to facilitate visualization using, for example, one or more of fluoroscopy, computer tomography (CT) fluoroscopy, or the like. The radiopaque portion can be a component of endovascular device 100. In one embodiment, at least one frame component 112 comprises a radiopaque material. Non-limiting examples of a radiopaque material include platinum or tantalum DFT Nitinol. Referring to FIG. 2, in another embodiment, a separate radiopaque marker is provided, such as radiopaque component 124 coupled at the junction where frame component 112 is coupled to the respective guide member, e.g., distal guide member 116. Endovascular device 100 can have one or more than one radiopaque marker coupled at various positions. For instance, each capture member 102, 104 can have its own radiopaque component 124.

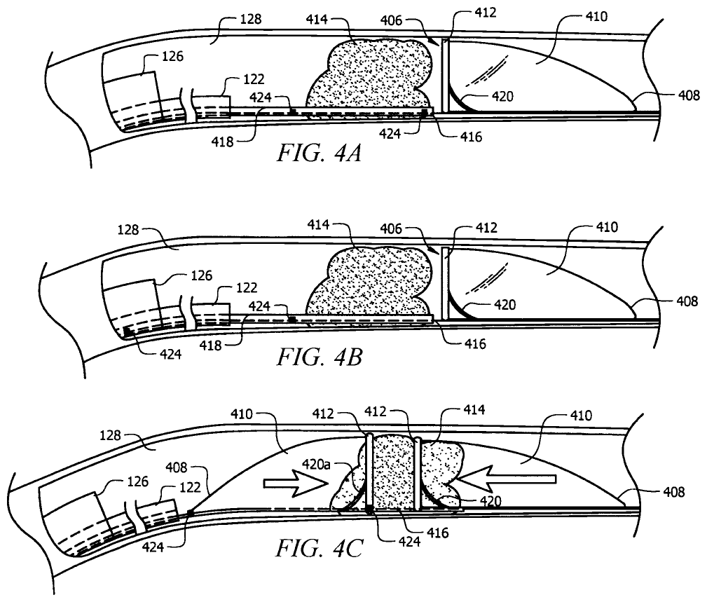

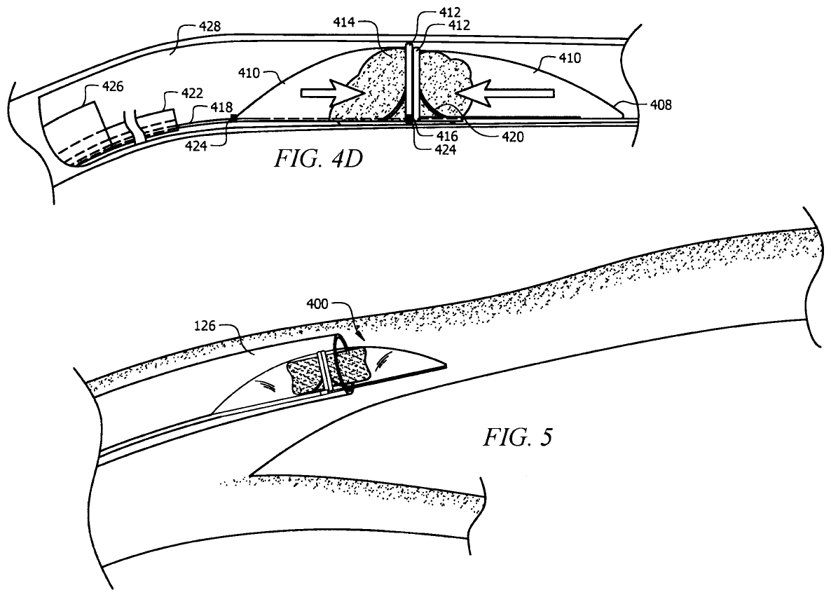

Referring to FIGS. 2-3, once all or substantially all of clot 114 is captured in the enclosure formed by capture members 102, 104, clot 114 can be removed by holding guide members 116, 118 together so they can remain united with one another as a unit and be withdrawn together with clot 114 contained therein. Referring to FIG. 2, capture members 102, 104 can be pulled through a stretch of blood vessels as a unit containing clot 114 before device 100 enters catheter 126, as shown in FIG. 4, for removal from the patient's body. In the embodiment shown, catheter 126 has a diameter that is larger than the diameter of frame components 112.

FIGS. 4A-4D and 5 illustrate another embodiment of the endovascular device of the present disclosure, endovascular device 400. In a preferred embodiment, endovascular device 400 is similar to endovascular device 100, except open end 406 of distal capture member 404 is smaller than open end 406 of proximal capture member 402. As shown, in one embodiment, the outer diameter of frame component 412 of distal capture member 404 is smaller than the outer diameter of frame component 412 of the proximal capture member 402. This configuration can help to reduce any opening or gap that can form between both frame components 412 when they unite with one another. Other features discussed herein with respect to endovascular device 100, such as dimensions, materials, strand density, strand diameter, shape, position with respect to the blood vessel interior wall, coupling of guide members, radiopaque marker, etc., are also applicable to endovascular device 400, and thus need not be repeated.

Referring to FIG. 5, once all or substantially all of clot 414 is captured in the enclosure formed by capture members 402, 404, and clot 414 can be removed by holding guide members 416, 418 together so they can remain united with one another as a unit and be withdrawn together with clot 114 contained therein. The united capture members 402, 404, along with clot 414 can be pulled into catheter 424 for removal.

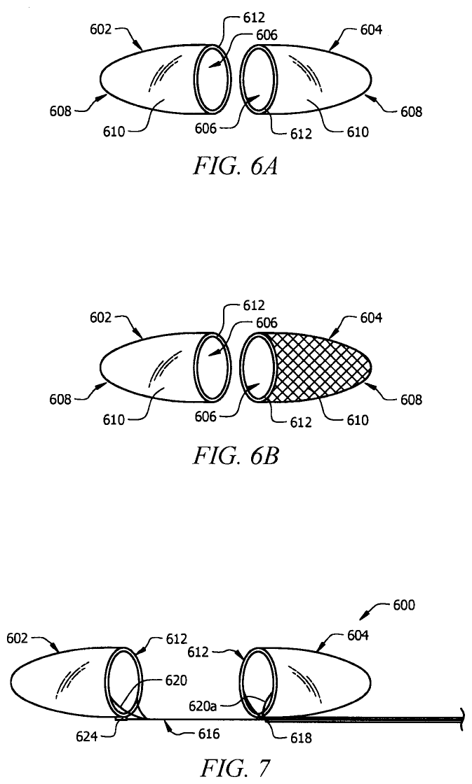

FIGS. 6A-6B, 7, and 8A-8F illustrate another embodiment of the endovascular device of the present disclosure, endovascular device 600. In a preferred embodiment, endovascular device 600 is similar to endovascular device 100, except for several features. In the embodiment shown in FIGS. 6A-6B, 7, and 8A-8F, tapered end 608 of capture members 602, 604 are not coupled to the respective guide members 616, 618. Referring to FIG. 6B, in another embodiment, body component 610 comprises polymeric net-like materials having a plurality of fenestrations throughout the material, such as, but not limited to, a woven mesh of polymeric material, metal, and/or other superelastic, self-expanding, and/or memory shape alloy such as nitinol. In certain embodiments, the woven mesh can comprise a combination of polymers, metals, and/or metal alloys. Referring to FIG. 7, one end of support arm 620 is coupled to distal guide member 616 in a manner that allows it to extend in the distal direction when capture member 602 is released from catheter 122. Likewise, in the embodiment shown, one end of support arm 620a is coupled to proximal guide member 618 in a manner that allows it to extend in the proximal direction when proximal capture member 604 is released from catheter 122. Endovascular device 600 can have any number of support arms 620, 620a. Further, support arms 620, 620a can be slidably coupled to the respective guide members 616, 618 as described above with respect to support arms 120, 120a. Other features discussed herein with respect to endovascular device 100, such as dimensions, materials, strand density, strand diameter, shape, position with respect to the blood vessel interior wall, coupling of guide members, radiopaque marker, etc., are also applicable to endovascular device 600, and thus need not be repeated.

According to another aspect of the present disclosure, there is a method of removing one or more material deposits in a lumen, such as a clot in a blood vessel, using embodiments of the endovascular device of the present disclosure, such as device 100, device 400, device 600, or device 900. While the disclosure may refer to numerical components of only one of device 100, 400, 600, 900, it is understood that the discussion is applicable to other unmentioned device and its components, such as those where combinations of embodiments comprise the device. In one embodiment, an endovascular device according to aspects of the present disclosure, e.g., device 100, 400, 600, or 900, configured to match the conditions, e.g., dimensions and shape, of the material deposit to be removed and the corresponding lumen conditions is selected.

Referring to FIGS. 8A, catheter 122 is provided to deliver endovascular device 100, 400, 600, or 900 to the site of the obstruction, or clot 114, in lumen 128. Catheter 122 can be referred to as a delivery catheter. In one embodiment, catheter 122 is a fluoroscopy microcatheter so visualization methods known to those skilled in the art, such as fluoroscopy, can be used to assist in delivering catheter 122 to the desired location. Catheter 122 is inserted into a patient's vessel and moved to clot 114 using means known to those skilled in the art, such as using another catheter, guide catheter 126, as shown in FIGS. 1A-1C. In such an embodiment, catheter 122 containing the endovascular device is advanced through the patient's body in guide catheter 126. As catheter 122 approaches clot 114, it naturally gravitates near the inner wall of lumen 128. In an embodiment, tip portion 130 of catheter 122 is moved distally through clot 114 to place tip portion 130 at a position distal to clot 114. In an embodiment, catheter 122 navigates to clot 114 without endovascular device 100, 400, 600, or 900 therein; however, catheter 122 with endovascular device 100, 400, 600, or 900 can travel to clot 114 together. After catheter 122 is at a desired position, endovascular device 100, 400, 600, or 900 is inserted into the lumen of catheter 122 in a compressed or collapsed configuration and can be moved through catheter 122 to arrive at clot 114. In a preferred embodiment, endovascular device 100, 400, 600, or 900 comprises flexible material that allows it to conform to catheter 122 as it makes its way through potentially tortuous paths without sustaining damage.

In an embodiment, when the distal end of endovascular device 100, 400, 600, or 900 approaches tip portion 130, device 100, 400, 600, or 900 is stabilized or steadied by manipulating the respective guide members (e.g., 116, 118, 910, 912) to place the respective capture members (e.g., 102, 104, 902, 904) in the desired positions and holding the guide members (e.g., 116, 118, 902, 904) together in place to maintain those positions. Referring to FIGS. 4A-4B, 8B, 9 and 14, the catheter (e.g. 122, 918) is then slowly withdrawn to release or unsheathe the distal capture member (e.g., 104, 404, 604, or 904) at a position distal to an obstruction. When released, the distal capture member (e.g., 104, 404, 604, or 904) expands to gently touch the inner lining of the vessel wall to open the respective body member (e.g., 110, 410, 610, or 914) to receive clot material. Non-limiting exemplary manners of expansion, such as through self-expanding material or mechanical expansion, including using inflatable members, are described above. In a preferred embodiment, the expansion of the distal capture member (e.g., 104, 404, 604, or 904) is preferably achieved with the expansion of its frame component (e.g., 112 412, 612, or 906), and/or body component (e.g., 110, 410, 610, or 914) to the original or expanded configuration. The distal guide member (e.g., 116, 416, 616, 912) is preferably stabilized, steadied, or held in place to maintain the distal capture member (e.g., 104, 404, 604, or 904) in the desired position distal to an obstruction. Catheter 122 and the proximal guide wire (e.g., 118, 418, 618, or 910) are then preferably held together so their movement are coupled to each other. Catheter 122 and the proximal guide wire (e.g., 118, 418, 618, or 910) are then preferably moved in the proximal position together as a unit to place the proximal capture member (e.g., 102, 402, 602, or 902) at a location proximal to an obstruction. Once the proximal capture member (e.g., 102, 402, 602, or 902) is in a desired location, the proximal guide member (e.g., 118, 418, 618, or 910) is then preferably coupled or held with the distal guide member (e.g., 116, 416, 616, or 912) to stabilize both capture members, maintaining them at the respective positions distally and proximally to an obstruction.

Next, referring to FIGS. 1A, 8C, 9, 13 and 14, the catheter (e.g. 122 or 918) is further withdrawn to unsheathe or release the proximal capture member (e.g., 102, 402, 602, or 902), which expands in a similar manner as the distal capture member (e.g., 104, 404, 604, or 904) as described above when released from the lumen of the catheter, to a position proximal to an obstruction. In one embodiment, the coupling angle between the frame component (e.g., 112, 412, or 612) and the respective guide member (e.g., 116, 118; 416, 418; 616, 618; 910, 912) can be increased and decreased by adjusting the position of the respective supporting arm (e.g., 120, 120a; or 420, 420a; or 620, 620a). This can be achieved by applying force to the fastening member slidably coupled to the respective guide member as described above in the desired direction, i.e., proximally or distally.

Referring to FIGS. 1B, 4C, 8D, 9, and 13, both guide members (e.g., 116, 118; 416, 418; 616, 618, or 910, 912) are manipulated to bring the capture members (e.g. 102, 104; 402, 404; 602, 604; or 902, 904) together. As shown in FIGS. 1B, 4C, 8D, 9, and 13, frame components (e.g., 112, 412, 612, or 906) of the capture members begin to engage the outer surface of the respective side of an obstruction. In one embodiment, the dislodging of an obstruction can be further aided by repetitively moving at least one capture member (e.g. 102, 104; 402, 404; 602, 604; or 902, 904) against an obstruction. This can be done by repetitive moving of the respective guide member itself and/or repetitive moving of the fastening member slidably coupled to that guide member. The effect is to gently separate an obstruction from the wall of lumen 128 before pulling the capture members over separated an obstruction, which allows for easier encapsulation of an obstruction.

In one embodiment, proximal capture member (e.g., 104, 404, 604, or 904) remains in one position while the distal capture member (e.g., 102, 402, 602, or 904) is moved via manipulation of the distal guide member (e.g., 116, 416, 616, or 912) to engage the distal end of an obstruction and bring the obstruction into both capture members (e.g. 102, 104; 402, 404; 602, 604; or 902, 904). The distal guide member (e.g., 116, 416, 616, or 912) is preferably continued to be withdrawn or pulled until the frame component (e.g., 112, 412, 612, or 906) of the distal capture member (e.g., 102, 402, 602, 902) unites or engages with the frame component (e.g., 112, 412, 612, or 906) of the proximal capture member (e.g., 102, 402, 602, or 902). Alternatively, for embodiments using frame components (e.g., 112, 412, 612, or 906) of different diameters, the distal guide member (e.g., 116, 416, 616, or 912) is preferably continued to be withdrawn or pulled until the capture members (e.g. 102, 104; 402, 404; 602, 604; 902, 904) join one another. In another embodiment, the distal capture member (e.g., 104, 404, 604, or 904) is kept in place while the proximal capture member (e.g., 102, 402, 602, or 902) is pushed in the distal direction toward the distal capture member (e.g., 104, 404, 604, or 904) to engage the proximal end of an obstruction and bring the obstruction into both capture members (e.g. 102, 104; 402, 404; 602, 604; 902, 904) through manipulation of the proximal guide member (e.g., 118, 418, or 618). In yet another embodiment, both capture members (e.g. 102, 104; 402, 404; 602, 604; 902, 904) can be moved toward one another, through manipulations of both guide members (e.g., 116, 118; 416, 418; 616, 618; or 910, 912) to engage the respective side of an obstruction. If equipped with suction capabilities, suctioning force can be applied when desired to further help direct an obstruction into either capture member (e.g. 102, 104; 402, 404; 602, 604; 902, 904).

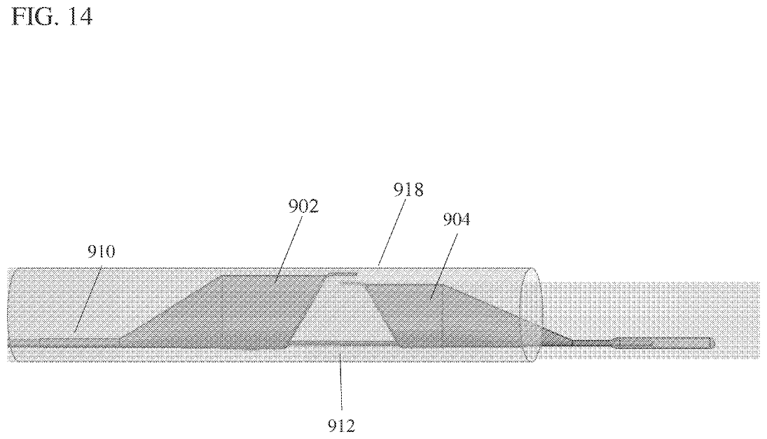

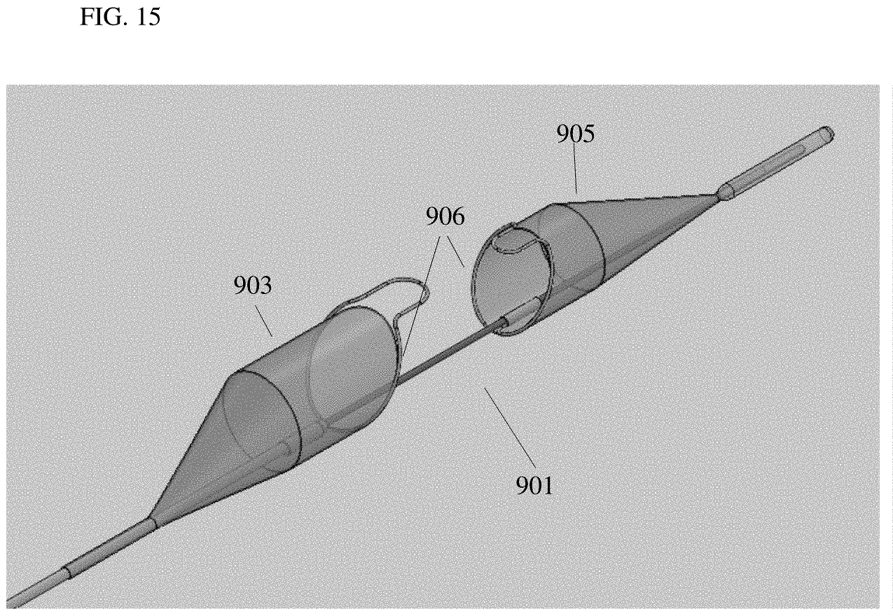

Referring to FIGS. 1C, 4D, and 8E, once all or substantially all of an obstruction is captured in the enclosure formed by capture members (e.g. 102, 104; 402, 404; 602, 604; 902, 904), an obstruction can be removed by holding guide members (e.g., 116, 118; 416, 418; 616, 618; or 910, 912) together so they can remain united with one another as a unit and be withdrawn together with an obstruction contained therein. Referring to FIG. 2, capture members (e.g. 102, 104; 402, 404; 602, 604; 902, 904) can be pulled through a stretch of blood vessels as a unit containing an obstruction before device 100, 400, 600, 900 enters the catheter (e.g. 126, 918), as shown in FIGS. 3, 5, and 14, for removal from the patient's body. As shown in FIG. 2, the flexibility of certain embodiments of the components according to certain aspects of the disclosure allow device 100, 400, 600, or 900 to conform to tortuous paths in a patient's body without inflicting additional damage as it is pulled out of the patient. Alternatively or in addition to, referring to FIGS. 8F and 14, device 100, 400, 600, or 900 along with an obstruction, can be withdrawn into a catheter (e.g. 122, 918) for removal, where the catheter (e.g. 122, 918) has a diameter smaller than the diameter of the frame component (e.g. 112, 906) in the expanded configuration, thereby compressing an obstruction.

Referring to FIG. 9, for device 900 the capture members 902 and 904 each have an opposing open end, comprising a frame component 906, slanted toward the other capture member. The device has guide member 910 and 912. The frame component 906 may be in fixed predetermined angle or in an adjustable angle relative to guide member 910 or 912.

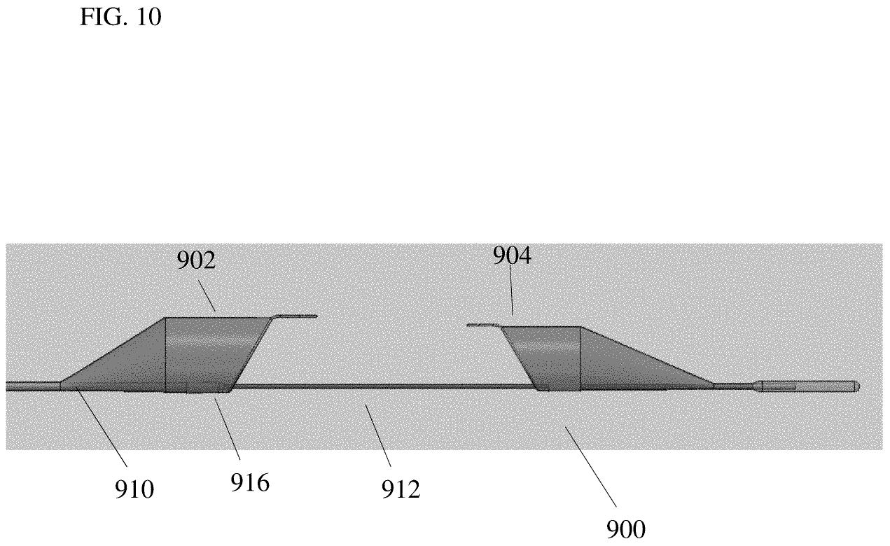

In some embodiments, the angle may be greater than 90 degrees, such as between 90 degrees and 120 degrees. In some embodiments, the angle is 91 degrees, 92 degrees, 93 degrees, 94 degrees, 95 degrees, 96 degrees, 97 degrees, 98 degrees, 99 degrees, 100 degrees, 101 degrees, 102 degrees, 103 degrees, 104 degrees, 105 degrees, 106 degrees, 107 degrees, 108 degrees, 109 degrees, 110 degrees, 111 degrees, 112 degrees, 113 degrees, 114 degrees, 115 degrees, 116 degrees, 117 degrees, 118 degrees, 119 degrees, or 120 degrees. In some embodiments, the angle of attachment of a first frame component 906 to guide member 910 is similar to angle of attachment of a second frame component 906 to guide member 912 both in degrees and direction. In other embodiments, the angle of attachment of a first frame component 906 to guide member 910 is different from angle of attachment of a second frame component to guide member 912 both in degrees and/or direction. In an embodiment, frame component 906 attached to guide member 910 and frame component 906 attached to guide member 912 are both angled toward each other, as shown in FIG. 10, giving the open ends at both distal and proximal capture members a slanted appearance toward an obstruction. In other embodiments, an open end of a capture member is slanted toward the corresponding clot end while the open end of the other capture member is slanted away from the corresponding clot end, as shown in FIG. 15.

Frame component 906 may have a measurement, such as a diameter and/or height, where the diameter may be any measurement going through the center of frame component 906. The height may be measured perpendicular from where the capture member (e.g. 902 or 904) is connected to the guide member (e.g. 910 or 912) to a point parallel with leading notch 908. In an embodiment, such as for an arterial clot, the diameter of capture member 902 and/or capture member 904 is approximately 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, or larger. In an embodiment, such as for venous clots, the diameter of capture member 902 and/or the member 904 is approximately 5 mm, 5.5 mm, 6 mm, 6.5 mm, 7 mm, 7.5 mm, 8 mm, 8.5 mm, 9 mm, 9.5 mm, 10 mm, 10.5 mm, 11 mm, 11.5 mm, 12 mm, 12.5 mm, 13 mm, 13.5 mm, 14 mm, 14.5 mm, 15 mm, 15.5 mm, 16 mm, 16.5 mm, 17 mm, 17.5 mm, 18 mm, 18.5 mm, 19 mm, 19.5 mm, 20 mm or larger. In certain embodiments, the measurements of frame component 906 are fixed and may be preselected to match the circumference of the intended lumen. In other embodiments, measurements of frame component 906 are slightly larger than the circumference of the intended lumen, including for example, between 0.1-1 mm.

In some embodiments, each frame component expands by at least one self expanding ring frame. As shown in FIG. 9, embodiments of the disclosure comprise a device wherein frame component 906 has leading notch 908, which may be parallel to the lumen (such as a vessel wall), and is designed to act as leading edge to first engage and dislodge an obstruction for optimal encapsulation. The leading notch 908 may have a fixed or adjustable angle. In some embodiments, leading notch 908 has a fixed angle of attachment to frame component 906 from which it arises in parallel axis to guide member 910 or 912. Leading notch 908 can have a 2D shape including but not limited to semicircular, semioval, horse shoe, rectangular, angular or any other suitable atraumatic shape and a 3D shape with convexity that follow the contour of the vessel or frame component 906 from which it arises.

Measurements of leading notch 908 include width along a line at the base of the notch in continuum with frame component 906 and depth along perpendicular line from the center of the base to the apex of the leading notch. Depth and width measurement are approximately 1×1 mm, 1.1×1 mm, 1.2×1 mm, 1.3×1 mm, 1.4×1 mm, or 1.5×1 mm. In general, the measurements of leading notch 908 are selected depending on the size the corresponding frame component 906 with width ideally between 10-30% of the circumference of frame component 906, and depth ideally between 50-150% of the width.

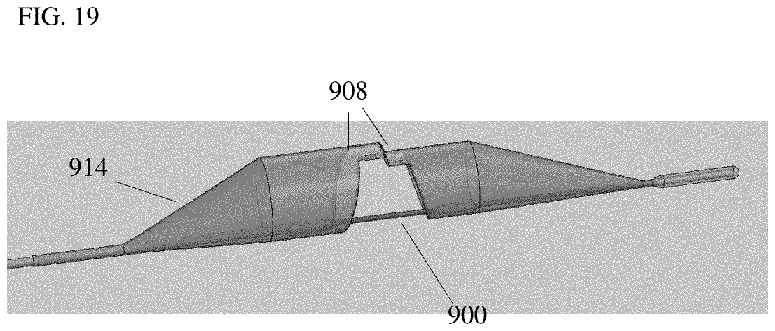

For each capture member 902 and 904, body component 914 attaches to at least a portion of the frame component 906 and guide member 910 and 912, respectively. As show in FIG. 9, leading notch 908, in some embodiments, is not covered by body component 914. As show in FIG. 19, leading notch 908, in some embodiments, is covered by body component 914. A surface of body component 914 may contain material to enhance the movement of its surface over the surface of the lumen and/or the obstruction. The material may be a hydrophilic material or coating

Referring to FIG. 10, the device 900 has the frame components of capture members 902 and 904 slanted in opposing directions. Frame component 906 of capture member 904 is angled proximally toward an obstruction while frame component 906 of capture member 902 is angled distally toward an obstruction giving the open ends a slanted configuration toward the obstruction when deployed as shown in FIG. 10. As seen in FIG. 10, certain embodiments have guide member 910 and guide member 912 slidably coupled. The embodiment depicted in FIG. 10 shows guide member 912 coupled into guide member 910 at 916, allowing guide member 912 to slide proximally and distally from guide member 910. FIG. 10 also shows an embodiment wherein the guide member 912 extends beyond capture member 904 and is capped by a capping component.

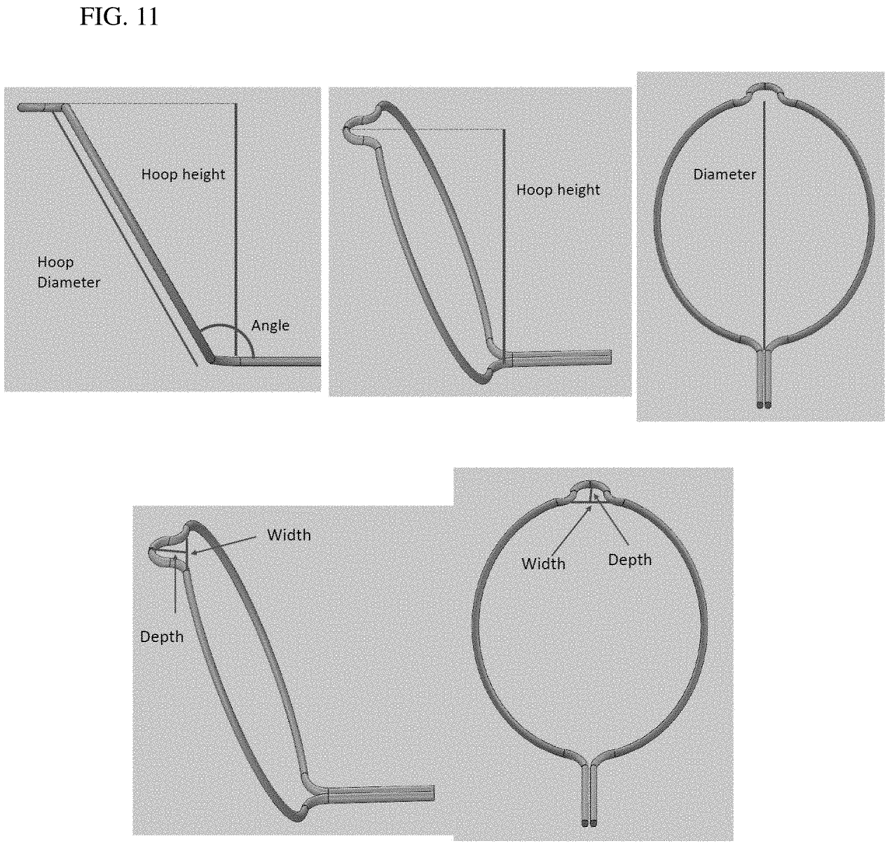

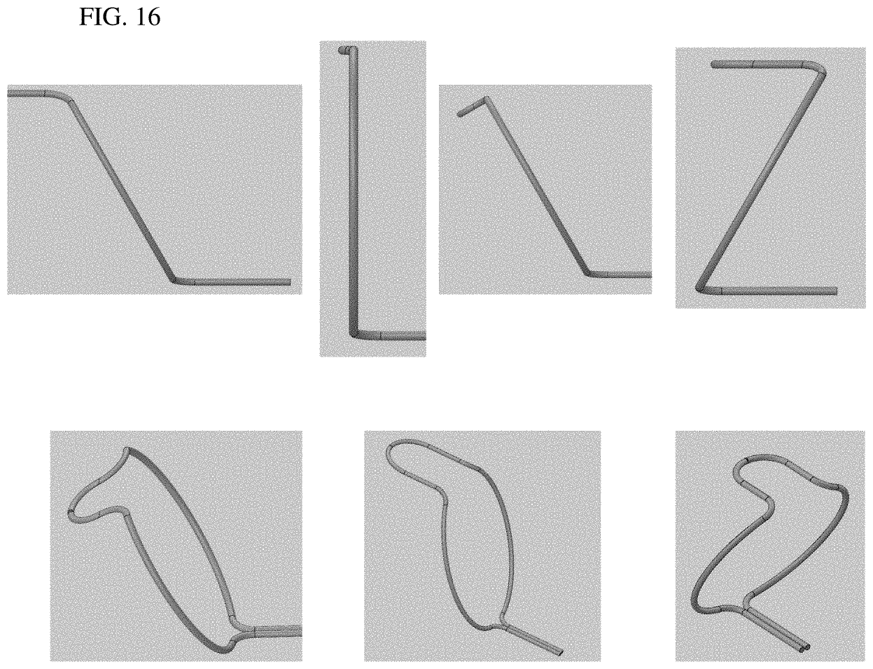

Referring to FIG. 11, frame component 906 and leading notch 908, of either capture member 902 or 904, may be configured in a number of different shapes at a number of different angles, including those shown in FIG. 16. Frame component 906 and leading notch 908 may be a variety of sizes, and frame component 906 and leading notch 908 of capture member 902 may be a different size from frame component 906 and leading notch 908 of capture member 904. Frame component 906 may have a hoop height and/or hoop diameter of any size capable of performing the function for which the devices of the disclosure are designed to accomplish. In some embodiments, the hoop height and/or hoop diameter is approximately 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, or larger. In some embodiments, leading notch 908 is approximately 1×1 mm, 1.1×1 mm, 1.2×1 mm, 1.3×1 mm, 1.4×1 mm, or 1.5×1 mm.

In some embodiments, frame component 906 of either capture member is at a non-perpendicular angle to guide member 910 or guide member 912. The angle may be greater than 90 degrees, such as between 90 degrees and 120 degrees. In some embodiments, the angle is 91 degrees, 92 degrees, 93 degrees, 94 degrees, 95 degrees, 96 degrees, 97 degrees, 98 degrees, 99 degrees, 100 degrees, 101 degrees, 102 degrees, 103 degrees, 104 degrees, 105 degrees, 106 degrees, 107 degrees, 108 degrees, 109 degrees, 110 degrees, 111 degrees, 112 degrees, 113 degrees, 114 degrees, 115 degrees, 116 degrees, 117 degrees, 118 degrees, 119 degrees, or 120 degrees.

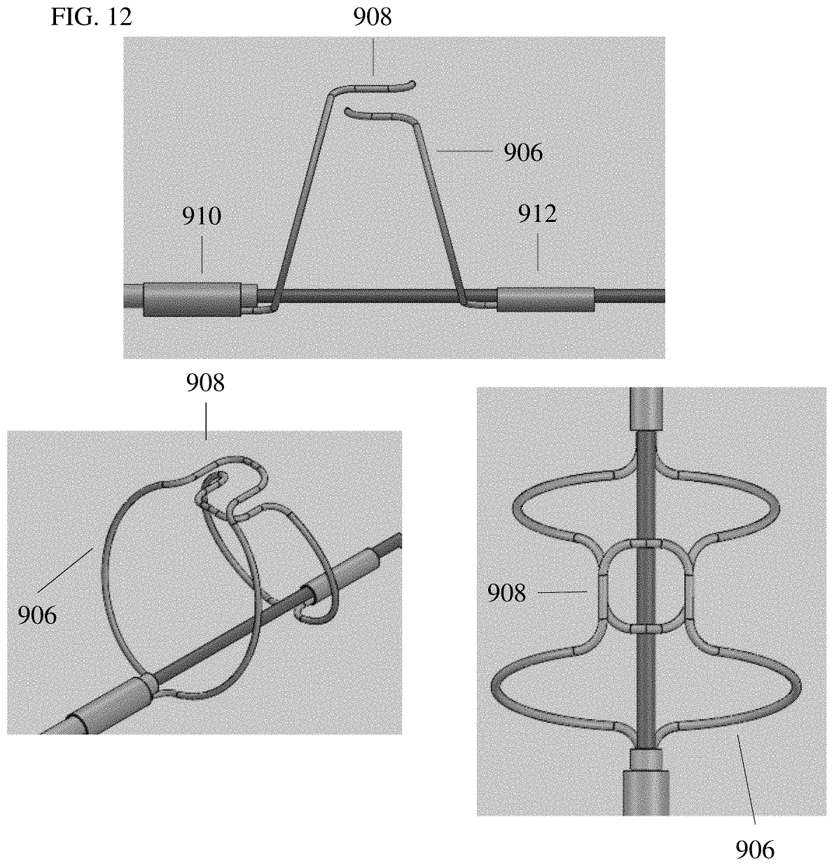

Referring to FIG. 12, certain devices of the disclosure comprising leading notch 908 on both frame components 906 are configured to lock the frame components by interlocking the leading notches. Frame component 906 of one capture member may be slightly larger than frame component 906 of the other capture member to facilitate interlocking. The leading notch interlocking may occur when one capture member is moved into the leading notch of the other capture member. In some embodiments, the capture member is moved by sliding guide members 910 and 912 relative to each other.

In certain embodiments, such as for tapering a blood vessel, the diameter of frame component 906 of the distal capture member (e.g. 904) is smaller than the diameter of frame component 906 of the proximal capture member (e.g. 902) and is configured to be retrieved inside frame component of the proximal capture member by manipulation of the guide members (e.g. 910 and 912) to form a tight seal enclosure around an obstruction and unite the capture members (e.g. 902 and 904) in an interlocking fashion. In other embodiments, frame component 906 of the distal capture member (e.g. 904) and at least a portion of body component 914 are configured to be retrieved inside frame component 906 and at least a portion of the body component 914 of the proximal capture member (e.g. 902) by manipulation of the guide members (e.g. 910 and 912) to form a tight seal enclosure around an obstruction and unite the capture members (e.g. 902 and 904) in an interlocking fashion. Interlocking of the capture members (e.g. 902 and 904) may involve the withdrawal of leading notch 908 on the distal capture member (e.g. 904) partially or fully underneath leading notch 908 of the proximal capture member (e.g. 902) and or the pushing of leading notch 908 of the proximal capture member (e.g. 902) partially or fully over leading notch 908 of the distal capture member (e.g. 904). Interlocking of the capture members (e.g. 902 and 904) may additionally involve the withdrawal of frame component 906 of the distal capture member (e.g. 904) partially or fully underneath frame component 906 of the proximal capture member (e.g. 902) and the pushing of frame component 906 of the proximal capture member (e.g. 906) partially or fully over frame component 906 of the distal capture member (e.g. 904). Additionally, interlocking of the capture members may involve the withdrawal of the both leading notch 908 and frame component 906 of the distal capture member (e.g. 904) underneath and proximal to both leading notch 908 and frame component 906 of the proximal capture member (e.g. 902) and/or the pushing of both leading notch 908 and frame component 906 of the proximal capture member (e.g. 902) over and distal to the both leading notch 908 and frame component 906 of the distal capture member (e.g. 904) to form a tight seal around an obstruction.

As seen in FIG. 12, the apex of leading notch 908 may be raised slightly to facilitate interlocking and to prevent the capture members from slipping apart after the obstruction is encapsulated. The leading notches may either completely or partially lock.

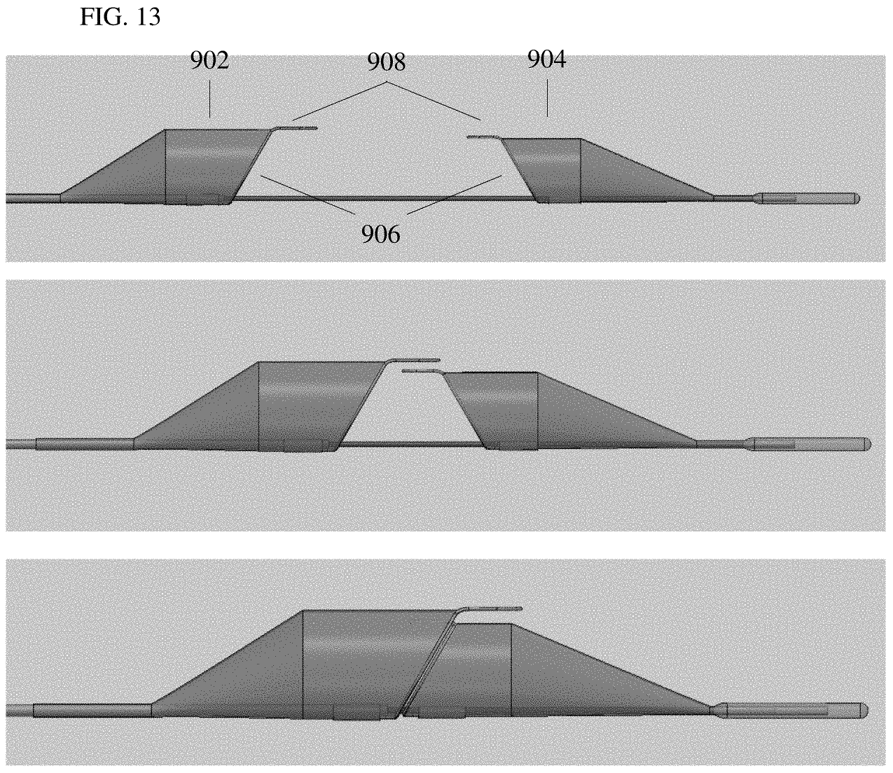

Referring to FIG. 13, capture members 902 and 904 may interact via of the leading notches 908 and the angled frame components 906 of each capture member while unseating and dislodging an opposing obstruction for optimal smooth enclosing around the obstruction surfaces without exerting significant stress on the obstruction or lumen/vessel walls. The bottom panel of FIG. 13 shows the eventual locking, such as by twisting, of capture member 904 into the open end of proximal capture member 904 encapsulating and isolating the obstruction from its surrounding environment. In preferred embodiment once the capture members (e.g. 902 and 904) are interlocked and united in a tight seal around an obstruction, the capture members (e.g. 902 and 904) are locked together with the obstruction trapped inside in this configuration during the removal process by pining the two guide member (e.g. 910 and 912) firmly together. In some embodiments, the guide members (e.g. 910 and 912) are locked in place using a hand held docking station attach to both guide members with a locking mechanism.

Referring to FIG. 14, devices disclosed herein, and methods using the devices, may be configured for the smooth retrieval of the capture members that have interlocked leading notches using a guide catheter 918. In one embodiment, retrieving the united interlocked capture members 902 and 904 and trapped obstruction involve first retrieving the tapered end and body component of proximal capture member 902 inside the lumen of guide catheter 918 with the mouth of guide catheter 918 pushing the distally facing frame component and leading notch of capture member 902 down toward guide member 910 which in turn push the underneath interlocked proximally facing frame component and leading notch of capture member 904 toward guide member 912 thus allowing the mouth of guide catheter 918 to smoothly glide over the overlapping frame components and leading notches without getting caught against the mouth of guide catheter 918 or interfere with the smooth full retrieval of united, interlocked capture members 902 and 904, with the trapped obstruction, inside the lumen of the guide catheter and eventually out of the body. In some embodiments, the mouth of guide catheter 918 pushes capture member 902 and its leading notch (which is facing away in relation to the mouth of guide catheter 918) to collapse distally and down, which will push capture member 904 down and allow the mouth of guide catheter 918 to smoothly glide over the opposing leading notch of capture member 904 allowing capture member 904 and its frame component to be pulled and retrieved inside guide catheter 918 with ease while preventing the leading notch of capture member 904 from getting caught against the mouth of guide catheter 918 or shaving away part of an obstruction that is encapsulated in the device.

Referring to FIG. 15, device 901 has capture members 903 and 905 with frame components 906 angled in the same direction resulting in both frame components 906 being parallel to each other.

Referring to FIG. 16, the frame components and leading notches of any of the capture members may have different shapes or configurations. Certain devices are configured to be capable of being manually manipulated to adjust the angles of engagement, including the angle of engagement with an obstruction and/or to mechanically separate an adherent obstruction from a vessel wall. In some embodiments, the obstruction is separated through a joggling motion on the surface of the obstruction and/or vessel wall.

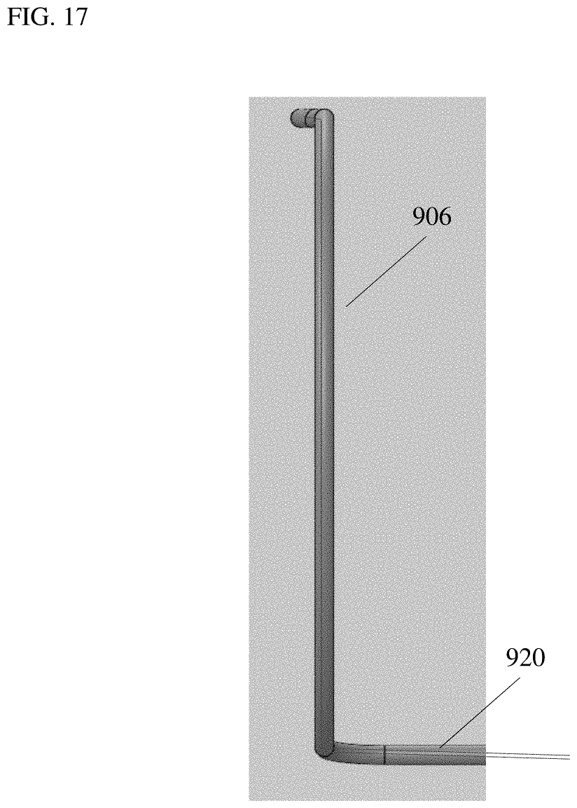

FIG. 17 shows an example of mechanism to manually manipulate and adjust the angels of the notch and/or frame component 906 through one or more microcables 920 attached around one or more joints at the base of the leading notch and/or frame component 906. Mircocable 920 may be run through either guide member 910 or 912 and may be attached to frame component 906 or leading notch 908. In some embodiments, microcable 920 is attached at a point that, when microcable 920 is manipulated, the angle of frame component 906 or the leading notch is adjusted.

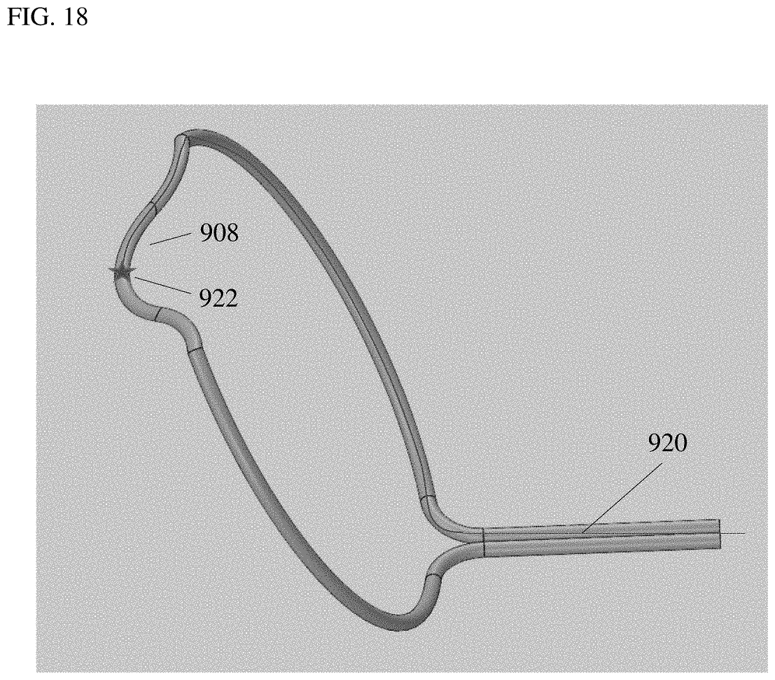

Referring to FIG. 18, certain devices comprise at least one microcable 920 running along or through a guide member and frame component to the apex of leading notch 908. In some embodiments, the apex of leading notch 908 comprises least one micro-transducer 922. Micro-transducer 922 may be configured for ultrasound imaging or optical tomography to enhance the visualization of the obstruction and site of impaction. This may allow for adjustments in the angle of engagement. The micro-transducer 922 can be fed by at least one of microcables 920.

Referring to FIG. 20, in embodiments with body component 914 made of flexible polymer, such as PET or nylon, full expansion of the body component can be aided by one or more wires of self expanded nitinol material couple to the inner surface of the body in longitudinal, circular helical or loosely braided configuration and any combination thereof.

As described, certain embodiments of the present disclosure provide for an endovascular device containing less overall metal material, making the device more flexible with smaller profile, which is particularly applicable to ease of navigation in small and torturous environments, such as brain circulation. Certain embodiments with less metallic material also provide less trauma to the lining of the small and fragile brain blood vessels during insertion and removal. The shape and size of the capture members of certain embodiments allow for better entrapment of the obstruction without significant compression of deformation which mean less fragmentation or pushing into normal side branch. In certain embodiments, the coupling of the frame component to the respective guide member leaves the open end of the capture member unobstructed, giving more space for the clot material to enter the capture member.

Although the embodiments of the present disclosure and their advantages have been described in detail, it should be understood that various changes, substitutions and alterations can be made herein without departing from the spirit and scope of the invention as defined by the appended claims. Embodiments, and elements of certain embodiments, may be combined or interchanged with other embodiments, or elements of embodiments, to make any device comprising one or more elements disclosed herein. Disclosed herein also includes methods for using any of the devices encompassed herein. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A device to remove an obstruction in a lumen comprising:
a. a first capture member;
b. a second capture member;
wherein the first capture member and the second capture member each comprise an open end comprising a frame component with at least one leading notch, a tapered end, and a body component extending between the open end and the tapered end; wherein the leading notch in the first capture member frame component is capable of interlocking with the leading notch in the second capture member frame component;
c. a first guide member directly connected to the first capture member; and
d. a second guide member directly connected to the second capture member, wherein the first guide member and the second guide member are slidably coupled;
wherein the frame component of the first capture member and the frame component of the second capture member are not perpendicular to the first guide member.

2. The device of claim 1, wherein the device is configured to fit within a catheter for deliver to said obstruction.

3. The device of claim 1, wherein at least one body component comprises a woven material of at least one of the following: a polymer, a metal, and any combination thereof.

4. The device of claim 1, wherein the frame component of the first capture member and/or the frame component of the second capture member are at an angle greater than 90 degrees relative to the guide member.

5. The device of claim 1, wherein the frame component of the first capture member and/or the frame component of the second capture member are at an angle between 90 degrees and 120 degrees relative to the guide member.

6. The device of claim 1, wherein the frame component of the first capture member and/or the frame component of the second capture member are at approximately 105 degree angle relative to the guide member.

7. The device of claim 1, wherein the first capture member and/or the second capture member are self-expanding.

8. The device of claim 1, wherein the frame component of the first capture member has a height and/or diameter of approximately 3.5 mm.

9. The device of claim 1, wherein the frame component of the second capture member has a height and/or diameter of approximately 3 mm.

10. The device of any one of claims 1-9 claim 1, wherein the frame component of the first capture member has a height and/or diameter of approximately 3.5 mm and wherein the frame component of the second capture member has a height and/or diameter of approximately 3 mm.

11. The device of claim 1, wherein the leading notch on the first capture member is approximately 1.2 mm×1 mm.

12. The device of claim 1, wherein the leading notch on the second capture member is approximately 1 mm×1 mm.

13. The device of claim 1, wherein the leading notch on the first capture member is approximately 1.2 mm×1 mm and the leading notch on the second capture member is approximately 1 mm×1 mm.

14. The device of claim 1, further comprising at least one microcable capable of manipulating an angle of the frame component.

15. The device of claim 1, further comprising a micro-transducer.

16. The device of claim 15, wherein the micro-transducer is capable of producing a signal for ultrasound imaging and/or optical tomography.

17. The device of claim 1, further comprising a radiopaque material.

18. The device of claim 17, wherein the radiopaque material is attached to the leading notch, the frame component, and/or the guide member of either or both capture members.

19. The device of claim 17, wherein either or both frame components and/or either or both leading notches are comprised of the radiopaque material.

20. A device to remove an obstruction in a lumen comprising:
a. a first capture member;
b. a second capture member;
wherein the first capture member and the second capture member comprise a body component and at least one leading notch; and
c. a guide member, wherein the first capture member and the second capture member are connected to the guide member, wherein the first capture member and the second capture member are configured to mechanically separate the obstruction in the lumen by manipulating the first capture member and the second capture member with the guide member to contact the obstruction with the leading notch.

21. The device of claim 20, wherein the device is configured to encapsulate the obstruction within the body component of the first capture member and the second capture member.

22. The device of claim 20, wherein the first capture member and the second capture member comprise a leading edge that is at a non-perpendicular angle to the guide member.

23. The device of claim 20, wherein the leading notch of the first capture member and the leading notch of the second capture member are capable of interlocking.

24. The device of claim 23, wherein the interlocking occurs by pushing a leading notch on the first body component over a leading notch on the second body component or withdrawing a leading notch on the second body component under a leading notch on the first body component through manipulating the guide member.

25. (canceled)

26. (canceled)

27. (canceled)

28. (canceled)

29. (canceled)

30. (canceled)

31. (canceled)

32. (canceled)

33. (canceled)
